(19) United States
(12) Patent Application Publication
(10) Pub. No.: US 2025/0066811 A1
(43) Pub. Date: Feb. 27, 2025

(54) DNA Amplification Method Using CARE Elements

(71) Applicant: Oxford Genetics Limited, Oxford, Oxfordshire (GB)

(72) Inventors: Ryan Cawood, Oxford, Oxfordshire (GB); David Brighty, Oxford, Oxfordshire (GB); Adam Gillman, Oxford, Oxfordshire (GB); Weiheng Su, Oxford, Oxfordshire (GB)

(21) Appl. No.: 18/553,948

(22) PCT Filed: Apr. 14, 2022
(86) PCT No.: PCT/GB2022/050957

§ 371 Date: Oct. 4, 2023

(30) Foreign Application Priority Data
Apr. 19, 2021 (GB) 2105581.9
Jul. 29, 2021 (GB) 2110922.8
Sep. 23, 2021 (GB) 2113603.1
Nov. 17, 2021 (GB) 2116571.7

(51) Int. Cl. C12N 15/86; C12N 15/113

(52) U.S. Cl. CPC C12N 15/86; C12N 15/1131; C12N 2310/14; C12N 2510/00; C12N 2750/14143; C12N 2750/14151; C12N 2830/60

(57) ABSTRACT

The present invention relates to methods of amplifying a nucleotide sequence. The nucleotide sequence to be amplified is flanked by CARE elements. The invention provides nucleic acid molecules (e.g. plasmids and vectors) comprising first and second CARE elements, flanking the nucleotide sequence to be amplified. The invention also provides host cells comprising such nucleic acid molecules and methods of amplification using such nucleic acid molecules. The invention is particularly applicable to the amplification of viral genes and the production of recombinant adeno-associated viruses (AAVs).

### CROSS-REFERENCE

This application is a section 371 U.S. national phase of PCT/GB2021/050957, filed Apr. 14, 2022, which claims priority from GB 2105581.9, filed Apr. 19, 2021; GB 2110922.8, filed Jul. 29, 2021; GB 2113603.1, filed Sep. 23, 2021; and GB 2116571.7, filed Nov. 17, 2021, all which are incorporated by reference in their entirety.

### STATEMENT REGARDING SEQUENCE LISTING

A computer readable form of the Sequence Listing is filed with this application by electronic submission and is incorporated into this application by reference in its entirety. The sequence listing submitted herewith is contained in the text file created Oct. 4, 2023, entitled “23-1436-WO-US_Sequence-Listing_ST25.txt” and 24,576 bytes in size.

### FIELD OF THE INVENTION

The present invention relates to methods of amplifying a nucleotide sequence. The invention provides nucleic acid molecules (e.g. plasmids and vectors) comprising first and second CARE elements, flanking the nucleotide sequence to be amplified. The invention also provides host cells comprising such nucleic acid molecules and methods of amplification using such nucleic acid molecules

### BACKGROUND OF THE INVENTION

It has previously been reported (Tessier, J., et al. J. Virol. 2001; 375-383; Chadeuf, G., et al., J. Gene Med. 2000; 2:260-268) that the transfection of Hela cell lines into which AAV rep and cap genes had been chromosomally integrated using AAV vectors and adenoviral infection lead to a 100-fold amplification of the integrated rep-cap sequence. These amplified sequences were present in an extrachromosomal form. An adenovirus DNA-binding protein (DBP) was said in this paper to be essential for this amplification.

This phenomenon was further described in US2004/0014031 in which a single cis-acting replication element (CARE) was involved. This CARE element was said to be located in a 171 nucleotide region corresponding to nucleotides 190-361 of the AAV2 genome (Example 12); this region encompasses the AAV p5 promoter.

Additionally, it was reported in US2004/0014031 that the CARE element was capable of inducing the amplification of an adjacent heterologous gene (Example 11), i.e. a single CARE element was capable of acting as an origin of replication.

These reports raised the possibility of using the CARE element to amplify an operably-associated nucleotide sequence.

### SUMMARY OF THE INVENTION

It has now been found that a single CARE element is insufficient to promote CARE-dependent replication: two CARE elements are necessary; these must flank the nucleotide sequence to be amplified; and the two CARE elements must be positioned in the same relative orientation (5′ to 3′) in a DNA molecule. The identification of the precise mechanism of action of such CARE elements facilitates the production of novel methods of amplifying nucleotide sequences.

The present invention relates to methods of amplifying a nucleotide sequence. The nucleotide sequence to be amplified is flanked by CARE elements. The invention provides nucleic acid molecules (e.g. plasmids and vectors) comprising first and second CARE elements, flanking the nucleotide sequence to be amplified. The invention also provides host cells comprising such nucleic acid molecules and methods of amplification using such nucleic acid molecules. The invention is particularly applicable to the amplification of viral genes and the production of recombinant adeno-associated viruses (AAVs).

It is one object of the invention, therefore, to provide nucleic acid molecules (e.g. plasmids and vectors) comprising first and second CARE elements flanking a nucleotide sequence to be amplified, e.g. a region such as a gene of interest driven by a promoter. Further objects of the invention include providing a host cell comprising such nucleic acid molecules and methods of amplification using such nucleic acid molecules.

The invention is particularly applicable to the amplification of viral genes and the production of recombinant adeno-associated viruses (AAVs). It is thus a further object of the invention to provide a method of AAV production wherein high expression levels of Rep and/or Cap polypeptides are obtained to make AAV particles.

### DESCRIPTION OF THE DRAWINGS

FIG. 1 shows examples of three CARE amplification cassettes of the invention.

FIG. 2 shows the results of CARE-directed amplification in Hela cells using CARE amplification cassettes of the invention and controls.

FIG. 3 shows the use of siRNA targeting Ad5 E2B genes, and that this decreases replication of TERA-E1 and Ad5-E1 genomes in HeLaRC32 cells.

FIG. 4 shows the use of siRNA targeting the Ad5 E2B gene, and that this increases DNA amplification of AAV2 rep from HeLaRC32 cells infected with Ad5-E1 and also TERA-E1 during MLP-repression (DMSO group).

FIG. 5 shows the use of siRNA targeting Ad5 E2B unit, and that this increases DNA amplification of AAV2 cap from HeLaRC32 cells infected with Ad5-E1 and also TERA-E1 during MLP-repression (DMSO group).

FIG. 6 shows the use of a Rep78 expression plasmid to induce CARE-directed amplification in 293AD cells using CARE amplification cassettes of the invention and controls.

FIG. 7 shows the results of CARE-directed amplification in 293AD cells using CARE amplification cassettes of the invention and controls.

FIG. 8 shows the results of CARE-directed amplification in CHO-X cells using CARE amplification cassettes of the invention and controls.

FIG. 9 shows the results of CARE-directed amplification in 239AD cells using CARE amplification cassettes embodying the invention.

### DETAILED DESCRIPTION OF THE INVENTION

In one embodiment, the invention provides a method of amplifying a first nucleotide sequence in a host cell, the method comprising the step:
(a) culturing a host cell which comprises:
   (i) a first nucleic acid molecule comprising:
      (a) a first CARE element, wherein the first CARE element comprises or consists of an AAV p5 promoter;
      (b) a first nucleotide sequence; and
      (c) a second CARE element, wherein the second CARE element comprises or consists of an AAV p5 promoter;
   wherein (a), (b) and (c) are operably-associated in this order (5′-3′) in the nucleic acid molecule and wherein the first and second CARE elements are both in the same 5′-3′ orientation;
and optionally additionally
   (ii) one or more second nucleic acid molecules comprising one or more promoters operably-associated with one or more adenovirus Early genes or adenovirus Late genes,
under conditions such that AAV Rep and one or more adenovirus Early gene products and/or adenovirus Late gene products are present in the host cell, thus promoting the amplification of the first nucleotide sequence.

In a further embodiment, the invention provides a process for producing recombinant viral particles comprising a transgene, the process comprising the steps:
(a) culturing a host cell comprising:
   (i) a Transfer Plasmid comprising 5′- and 3′-viral ITRs flanking a transgene;
   (ii) a nucleic acid molecule of the invention, the first nucleotide sequence comprising viral rep and cap genes, the nucleic acid molecule either being present in an episomal plasmid or vector within the host cell or being integrated into the host cell genome; and
   (iii) sufficient AV helper genes for promoting amplification of the rep and cap genes and for packaging the Transfer Plasmid, the helper genes either being present in an episomal Helper Plasmid within the host cell, in an adenoviral vector or being integrated into the host cell genome;
under conditions such that viral rep and cap genes are amplified and viral particles are assembled by the host cell; and
(b) harvesting packaged viral particles from the host cells or from the culture medium.

In yet a further embodiment, the invention provides a nucleic acid molecule comprising:
(a) a first CARE element, wherein the first CARE element comprises an AAV p5 promoter;
(b) a first nucleotide sequence; and
(c) a second CARE element, wherein the second CARE element comprises an AAV p5 promoter;
   wherein one or both of the first and/or second CARE element independently additionally comprise a pre-AAV p5 promoter region,
   wherein (a), (b) and (c) are operably-associated in this order (5′-3′) in the nucleic acid molecule and wherein the first and second CARE elements are both in the same 5′-3′ orientation.

In a further embodiment, the invention provides a host cell comprising a nucleic acid molecule of the invention.

In a further embodiment, the invention provides a process for producing a modified host cell, the process comprising:
(a) introducing a nucleic acid molecule of the invention into a host cell, thereby producing a modified host cell.

The nucleic acid molecule of the invention is preferably DNA. It may be single-stranded or double-stranded.

As used herein, the term “CARE element” refers to a Cis-Acting Replication Element. The CARE element is a sequence of nucleotides which, in the presence of adenovirus and AAV Rep proteins, is capable of promoting the replication of a first nucleotide sequence, when the first nucleotide sequence is flanked by first and second such CARE elements, and wherein the first and second CARE elements are both in the same 5′-3′ orientation. Examples of CARE elements have previously been described by Tessier, J., et al. J. Virol. 2001; 375-383; Chadeuf, G., et al. J. Gene Med. 2000; 2:260-268; US2004/0014031; and Nony, P. et al. J. Virol. 2001. The CARE element is preferably an AAV CARE element.

The first and second CARE elements used in the invention each independently comprise an AAV p5 promoter. The p5 promoter sequences in the first and second CARE elements may be the same or different.

The wild-type AAV p5 promoter comprises:
(a) a MLTF/USF1 binding site,
(b) a YY1-60 binding site,
(c) a TATA box,
(d) an AAV Rep binding site,
(e) a trs element, and
(f) a YY1+1 binding site.

Preferably, the AAV p5 promoters in the first and second CARE elements of the invention independently comprise 1, 2, 3, 4, 5 or 6 of the above (a)-(f), more preferably 4-6 or 5-6 of (a)-(f), and most preferably all of the above (a)-(f). When present in the p5 promoter, each of (a)-(f) are preferably in the above-stated order (5′-3′).

The wild-type AAV p5 promoter promotes expression of Rep 78 and Rep 68 polypeptides. The p5 promoter is located at the 5′ end of the wild-type rep gene. The wild-type AAV2 p5 promoter has the nucleotide sequence as given in SEQ ID NO: 1:
gtcctgtattagaggtcacgtgagtgttttgcgacattttgcgacaccatgtggtcacgctgggtatttaa gcccgagtgagcacgcagggtctccattttgaagcgggaggtttgaacgcgcagccgcc (SEQ ID NO: 1).

In the above sequence, the MLTF/USF1 binding site, YY1-60 binding site, TATA box AAV Rep binding site, and YY1+1 binding site are underlined, in this order. The trs element falls within the YY1+1 binding site. The core promoter sequence is highlighted in bold.

In the CARE elements used in the invention, the p5 promoter is generally a functional p5 promoter. As used herein, the term “functional p5 promoter” refers preferably to a nucleotide sequence which consists of or comprises the nucleotide sequence of SEQ ID NO: 1 or a variant thereof having at least 80%, 85%, 90%, 95% or 99% sequence identity thereto and which is capable of promoting the transcription of an operably-associated first nucleotide molecule which encodes one or more AAV Rep polypeptides, preferably the Rep 78 and Rep68 polypeptides. The level of activity of a p5 promoter may be determined by operably-associating a test p5 promoter sequence with a suitable transgene and assaying for the level of expression of the transgene. A level of expression which is less than 5% (preferably less than 1%) of the expression level from a wild-type AAV p5 promoter when operably-associated with the same transgene may be considered to be not functional.

MLTF/USF1 is a member of the basic helix-loop-helix bHLH leucine zipper family. It is an ubiquitously expressed transcription factor which binds to DNA enhancer box response elements. The MLTF/USF1 binding site sequence is aggtcacgtgagtg (SEQ ID NO: 2). The invention encompasses variants of the aforementioned sequence (e.g. having 1, 2 or 3 nucleotide substitutions) which are capable of binding MLTF/USF1. It is thought that the MLTF/USF1 is important for CARE-based amplification, but it might not be essential.

YY1 (Yin-Yang 1) is a ubiquitously-distributed transcription factor belonging to the GLI-Kruppel class of zinc finger proteins. The protein is involved in repressing and activating a diverse number of promoters. The YY1-60 binding site sequence is cgacatttt. The invention encompasses variants of the aforementioned sequence (e.g. having 1, 2 or 3 nucleotide substitutions) which are capable of binding YY1. It is thought that the YY1-60 binding site is important for CARE-based amplification, but it might not be essential. Alternatively, the YY1+1 binding site may be used instead of the YY1-60 site.

The p5 TATA box has the sequence tatttaa. The p5 promoter's TATA box within the CARE element is thought to be required for CARE-based amplification. The invention encompasses variants of the aforementioned sequence (e.g. having 1, 2 or 3 nucleotide substitutions).

The Rep binding site (RBS) is also known as a Rep Recognition Sequence (RRS) or a Rep Binding Element (RBE). There are three RBSs in the AAV genome: one in each of the inverted terminal repeats (ITRs); and another one in a region termed the P5 integration efficiency element (P5IEE) that encompasses the p5 promoter.

The AAV RBS has the following sequence: gcccgagtgagcacgc (SEQ ID NO: 3). The 16-mer core sequence of the RRS in the AAV ITR (AAV-ITR-RBS) in given in SEQ ID NO: 4: 5′-GAGCGAGCGAGCGCGC-3′ (SEQ ID NO: 4). The 16-mer core sequence in the RBS in AAVS1 (AAVS1-RBS) is given in SEQ ID NO: 5: 5′-CAGCGAGCGAGCGAGC-3′ (SEQ ID NO: 5). The invention encompasses variants of the aforementioned sequences (e.g. having 1, 2 or 3 nucleotide substitutions) which are capable of binding an AAV Rep polypeptide, most preferably an AAV Rep78 polypeptide. The presence of an RBS site within the CARE element is thought to be required for CARE-based amplification.

The terminal resolution site (trs) is targeted by Rep78/68, which possess endonuclease activity, and is cleaved in a site- and strand-specific manner. This mechanism resolves the double stranded AAV molecules generated during replication into packageable single stranded virial genomes. In the AAV p5 promoter, the trs element has the sequence: ccattt The invention encompasses variants of the above sequence (e.g. 1, 2 or 3 nucleotide substitutions) which are capable of binding Rep78 or Rep 68. The trs element is possibly essential.

The YY1+1 binding site sequence is ctccatttt. The invention encompasses variants of the above sequence (e.g. having 1, 2 or 3 nucleotide substitutions) which are capable of binding YY1. It is thought that the YY1+1 binding site is important for CARE-based amplification, but it might not be essential. Alternatively, the YY1-60 binding site may be used instead of the YY1+1 site.

In some embodiments, the p5 promoter in the first CARE element is modified to prevent rep gene expression from the p5 promoter in the first CARE element, preferably by insertion of a repressor binding site in the p5 promoter in the first CARE element.

In other embodiments, the rep gene is operably-associated with a heterologous inducible or repressible promoter.

The first and/or second CARE element may independently comprises a pre-AAV p5 promoter region. The pre-AAV p5 promoter region sequences in the first and second CARE elements may be the same or different. As used herein, the term “pre-AAV p5 promoter region” refers to a stretch of contiguous nucleotides having a sequence which corresponds to the sequence of a region of DNA which is upstream of the AAV p5 promoter.

In the wild-type AAV2 genome, nucleotides 1-145 encode the AAV's left inverted terminal repeat (ITR); and nucleotides 191-320 relate to the p5 promoter. In the AAV2 genome, therefore, the pre-AAV p5 promoter region is a stretch of contiguous nucleotides which encompasses part or all of nucleotides 146-190.

In one embodiment, the pre-AAV p5 promoter region comprises or consists of:
(i) AAV2 nucleotides 146-190, or the corresponding nucleotides from a different AAV serotype (e.g. AAV5);
(ii) a nucleotide sequence having at least 80%, 90% or 95% sequence identity to (i); or
(iii) a fragment of (i), the fragment being at least 80%, 90% or 95% of the length of (i).

The function of the pre-AAV p5 promoter region is to enhance the degree of DNA amplification of the first nucleotide sequence compared to the degree of DNA amplification which is obtainable by using a corresponding control nucleic acid molecule of the invention but which does not comprise a pre-AAV p5 promoter region.

In the invention, the pre-AAV p5 promoter region is upstream (5′) of the AAV p5 promoter and is operably-linked to the AAV p5 promoter. Preferably, the pre-AAV p5 promoter region is contiguously joined to the AAV p5 promoter.

The AAV CARE element may also comprise a 5′ portion of the AAV rep gene, e.g.

(SEQ ID NO: 24)
atgccggggttttacgagattgtgattaaggtccccagcgaccttgacga
gcatctgcccggcatttctgacagctttgtgaactgggtggccgagaagg
aatgggagttgccgccagattctgacatggatctgaatctgattgagcag
gcacccctgaccgtggccgagaagctgcagcgcgactttctgacggaatg
gcgccgtgtgagtaaggccc.

The above sequence includes nucleotides 321-541 of the AAV2 genome (i.e. 221 nucleotides of the AAV2 rep gene). The corresponding sequence in a different AAV genome may also be used.

Preferably, the 5′ portion of the AAV rep gene consists of or comprises nucleotides 321-353 of the AAV2 genome (i.e. 33 nucleotides of the AAV rep gene), for example (AAV2) atgccggggttttacgagattgtgattaaggtc (SEQ ID NO: 25). The corresponding sequence in a different AAV genome may also be used. In some embodiments, the CARE element does not comprise a 5′ portion of the AAV rep gene.

In some embodiments of the invention (e.g. wherein the first nucleotide sequence comprises a rep gene), the 5′ portion of the AAV rep gene (in the CARE element) does not comprise any ATG codons (thus minimising expression of Rep polypeptides). For example, ATG codons may be mutated to ATA codons.

As used herein, the term “5′ portion of the AAV rep gene” preferably refers to a nucleotide sequence which consists of or comprises SEQ ID NO: 24 or 25, or a variant thereof having at least 80%, 85%, 90%, 95% or 99% sequence identity thereto.

In some preferred embodiments, the CARE element consists of or comprises one of the following:
a region of an AAV genome corresponding to nucleotides 146 to 320 of the AAV2 genome as shown in SEQ ID NO: 6:

(SEQ ID NO: 6)
ggaggggtggagtcgtgacgtgaattacgtcatagggttagggaggtcct
gtattagaggtcacgtgagtgttttgcgacattttgcgacaccatgtggt
cacgctgggtatttaagcccgagtgagcacgcagggtctccattttgaag
cgggaggtttgaacgcgcagccgcc

a region of an AAV genome corresponding to nucleotides 191 to 353 of the AAV2 genome as shown in SEQ ID NO: 7:

(SEQ ID NO: 7)
gtcctgtattagaggtcacgtgagtgttttgcgacattttgcgacaccat
gtggtcacgctgggtatttaagcccgagtgagcacgcagggtctccattt
tgaagcgggaggtttgaacgcgcagccgccatgccggggttttacgagat
tgtgattaaggtc

a region of an AAV genome corresponding to nucleotides 146 to 190 of the AAV2 genome as shown in SEQ ID NO: 8:

(SEQ ID NO: 8)
ggaggggtggagtcgtgacgtgaattacgtcatagggttagggag

a region of an AAV genome corresponding to nucleotides 191 to 542 of the AAV2 genome as shown in SEQ ID NO: 9:

(SEQ ID NO: 9)
gtcctgtattagaggtcacgtgagtgttttgcgacattttgcgacaccat
gtggtcacgctgggtatttaagcccgagtgagcacgcagggtctccattt
tgaagcgggaggtttgaacgcqcagccgccatgccggggttttacgagat
tgtgattaaggtccccagcgaccttgacgagcatctgcccggcatttctg
acagctttgtgaactgggtggccgagaaggaatgggagttgccgccagat
tctgacatggatctgaatctgattgagcaggcacccctgaccgtggccga
gaagctgcagcgcgactttctgacggaatggcgccgtgtgagtaaggccc
cg.

(The p5 promoter is underlined.)
a region of an AAV genome corresponding to nucleotides 191 to 320 of the wild-type AAV2 genome as shown in SEQ ID NO: 10:

(SEQ ID NO: 10)
gtcctgtattagaggtcacgtgagtgttttgcgacattttgcgacaccat
gtggtcacgctgggtatttaagcccgagtgagcacgcagggtctccattt
tgaagcgggaggtttgaacgcgcagccgcc

a region of an AAV genome corresponding to nucleotides 146 to 542 of wild-type AAV2 genome as shown in SEQ ID NO: 11:

ggaggggtggagtcgtgacgtgaattacgtcatagggttagggaggtcct
gtattagaggtcacgtgagtgttttgcgacattttgcgacaccatgtggt
cacgctgggtatttaagcccgagtgagcacgcagggtctccattttgaag
cgggaggtttgaacgcgcagccgccatgccggggttttacgagattgtga
ttaaggtccccagcgaccttgacgagcatctgcccggcatttctgacagc
tttgtgaactgggtggccgagaaggaatgggagttgccgccagattctga
catggatctgaatctgattgagcaggcacccctgaccgtggccgagaagc
tgcagcgcgactttctgacggaatggcgccgtgtgagtaaggccccg

a region of an AAV genome corresponding to nucleotides 146 to 353 of the wild-type AAV2 genome as shown in SEQ ID NO: 12:

ggaggggtggagtcgtgacgtgaattacgtcatagggttagggaggtcct
gtattagaggtcacgtgagtgttttgcgacattttgcgacaccatgtggt
cacgctgggtatttaagcccgagtgagcacgcagggtctccattttgaag
cgggaggtttgaacgcgcagccgccatgccggggttttacgagattgtga
ttaaggtc

Preferably, the first and/or second CARE element has the nucleotide sequence as given in any one of SEQ ID NOs: 6-12, or a variant thereof having at least 50%, 60%, 70%, 80%, 90% or 95% sequence identify thereto and which is capable of promoting the amplification of a first nucleotide sequence which is flanked by first and second such variants, and wherein the first and second variants are both in the same 5′-3′ orientation, in the presence of adenovirus and AAV Rep proteins.

In some embodiments, the first and/or second CARE element is a functional fragment of the nucleotide sequence as given in any one of SEQ ID NOs: 6-12, wherein the functional fragment is at least 80%, 90% or 95% of the length of the nucleotide sequence.

As used herein, the term “functional fragment” is a sequence of nucleotides which, in the presence of adenovirus and AAV Rep proteins, is capable of promoting the replication of a first nucleotide sequence, when the first nucleotide sequence is flanked by first and second such fragments, and wherein the first and second fragments are both in the same 5′-3′ orientation.

In any of the CARE elements, the relative positions, absolute positions and/or nucleotide sequences of:
(c) a TATA box,
(d) an AAV Rep binding site, and
(e) a trs element,
   in the CARE element are preferably maintained, and preferably also the relative positions, absolute positions and/or sequences of:
(a) a MLTF/USF1 binding site,
(b) a YY1-60 binding site, and
(f) a YY1+1 binding site.

The first and second CARE elements are placed upstream and downstream of the first nucleotide sequence, respectively, such that:
(a) the first CARE element;
(b) the first nucleotide sequence; and
(c) the second CARE element;
   are operably-associated in this 5′-3′ order in the nucleic acid molecule.

Additionally, the first and second CARE elements are both in the same 5′-3′ orientation. The 5′-3′ orientation of the CARE element is defined according to its natural (wild-type) environment.

The distance between the 3′-end of the first CARE element and the 5′-end of the second CARE element may, for example, be 1-5 Kb, 5-10 Kb, 10-15 Kb, 15-50 Kb or 50-100 Kb. In other embodiments (for example a CARE-GOI-CARE cassette comprising a gene of interest, GOI), this distance may be 5 to 1000 nucleotides, 5-500 nucleotides or 5-50 nucleotides. In some embodiments, this distance is less than 1000 nucleotides, less than 100 nucleotides or less than 50 nucleotides. In other embodiments, this distance is at least 50, 100 or 1000 nucleotides.

The first and second CARE elements may or may not have the same nucleotide sequences.

The first nucleotide sequence is a sequence of contiguous nucleotides. The first nucleotide sequence may, in general, be any nucleic acid molecule which is desired to be amplified.

The first nucleotide sequence (e.g. its coding sequence) may be in 5′-3′ orientation or 3′-5′ orientation. In other words, the 3′ end of the first CARE element may be joined to the 3′ end of the first nucleotide sequence.

In one embodiment, the first nucleotide sequence is a nucleotide sequence that encodes a gene of interest (GOI) or transgene, and optionally an operably-associated promoter (e.g. a polymerase II promoter). This promoter may be constitutive or inducible.

In some embodiments, the first nucleotide sequence comprises a transgene. The first nucleotide sequence may be a coding or non-coding sequence. It may be genomic DNA or cDNA. Preferably, the nucleotide sequence encodes a polypeptide or a fragment thereof.

In some embodiments, the first nucleotide sequence codes for a therapeutic polypeptide or a fragment thereof. Examples of preferred therapeutic polypeptides include antibodies, CAR-T molecules, scFV, BiTEs, DARPins and T-cell receptors. In some embodiments, the therapeutic polypeptide is a G-protein coupled receptor (GPCR), e.g. DRD1. In some embodiments, the therapeutic polypeptide is a functioning copy of a gene involved in human vision or retinal function, e.g. RPE65 or REP. In some embodiments, the therapeutic polypeptide is a functioning copy of a gene involved in human blood production or is a blood component, e.g. Factor IX, or those involved in beta and alpha thalassemia or sickle cell anaemia. In some embodiments, the therapeutic polypeptide is a functioning copy of a gene involved in immune function such as that in severe combined immune-deficiency (SCID) or Adenosine deaminase deficiency (ADA-SCID). In some embodiments, the therapeutic polypeptide is a protein which increases/decreases proliferation of cells, e.g. a growth factor receptor. In some embodiments, the therapeutic polypeptide is an ion channel polypeptide. In some preferred embodiments, the therapeutic polypeptide is an immune checkpoint molecule. Preferably, the immune checkpoint molecule is PD1, PDL1, CTLA4, Lag1 or GITR.

In some preferred embodiments, the first nucleotide sequence encodes a CRISPR enzyme (e.g. Cas9, dCas9, Cas12, or a variant or derivative thereof) or a CRISPR sgRNA.

In some embodiments, the first nucleotide sequence comprises a gene from a virus which is known to infect a mammal. Genes encoded within the first nucleotide sequence may encode polypeptides that are able to self-assemble into viral-like particles that may or may not be used as a vaccine. In one preferred embodiment, the first nucleotide sequence encodes a norovirus capsid protein.

In other embodiments, the first nucleotide sequence may encode one or more polypeptides known to induce an immune response in humans as a vaccine that can self-assemble into multimeric complexes. A preferred embodiment would be to encode the five genes required for the cytomegalovirus (CMV) pentameric complex; these include CMV gH/gL/UL128/UL130/UL131.

In other embodiments, the first nucleotide sequence may encode a protein known to induce an immune response in humans as a vaccine that does not self-assemble into viral like particles. A preferred embodiment would be to encode the Ebola F protein, Influenza F and H proteins or the Coronavirus S, E or M proteins.

In some embodiments, the first nucleotide sequence comprises a gene from a retrovirus, more preferably a lentivirus. Such genes include, but are not limited, to the Gag-Pol gene, the Rev gene, and the Env gene.

In some embodiments, the first nucleotide sequence comprises a gene from a rhabdovirus, more preferably a vesicular stomatitis virus (VSV). Such genes include, but are not limited, to the VSV glycoprotein gene (i.e. the VSV G gene).

In some embodiments, the first nucleotide sequence comprises genes required to make a viral packaging cell line that encodes genes that are required to assemble a gene therapy viral vector or encodes a gene therapy transfer vector.

In some embodiments, the first nucleotide sequence comprises genes required to make a viral producer cell line that encodes all the genes and a transfer vector that are required to produce a gene therapy vector.

In yet other embodiments, the first nucleotide sequence may comprise one or more genes for lentiviral vectors (e.g. Gag-pol, REV, VSV-G, RD114) or one or more genes for adenoviral vectors (e.g. Hexon, Fibre, Penton, pVII, or pVI).

In some embodiments, the first nucleotide sequence comprises a rep gene sequence and/or a cap gene sequence, or a fragment thereof. Preferably, the rep and cap genes are AAV genes.

In some embodiments, the first nucleic acid molecule or the first nucleotide sequence does not comprise a rep gene sequence, or a fragment thereof.

In other embodiments, the first nucleic acid molecule or the first nucleotide sequence does not comprise a cap gene sequence, or a fragment thereof.

In some embodiments, the first nucleotide sequence is a nucleotide sequence which encodes a polypeptide selected from the group consisting of luciferase, Herceptin heavy chain, Herceptin light chain, TGFβ1 and BMP2.

As used herein, the term “rep gene” refers to a gene that encodes one or more open reading frames (ORFs), wherein each of said ORFs encodes an AAV Rep non-structural protein, or variant or derivative thereof. These AAV Rep non-structural proteins (or variants or derivatives thereof) are involved in AAV genome replication and/or AAV genome packaging.

The wild-type rep gene comprises three promoters: p5, p19 and p40. Two overlapping messenger ribonucleic acids (mRNAs) of different lengths can be produced from p5 and from p19. Each of these mRNAs contains an intron which can be either spliced out or not using a single splice donor site and two different splice acceptor sites. Thus, six different mRNAs can be formed, of which only four are functional. The two mRNAs that fail to remove the intron (one transcribed from p5 and one from p19) read through to a shared poly-adenylation terminator sequence and encode Rep78 and Rep52, respectively. Removal of the intron and use of the 5′-most splice acceptor site does not result in production of any functional Rep protein—it cannot produce the correct Rep68 or Rep40 proteins as the frame of the remainder of the sequence is shifted, and it will also not produce the correct C-terminus of Rep78 or Rep52 because their terminator is spliced out. Conversely, removal of the intron and use of the 3′ splice acceptor will include the correct C-terminus for Rep68 and Rep40, whilst splicing out the terminator of Rep78 and Rep52. Hence the only functional splicing either avoids splicing out the intron altogether (producing Rep78 and Rep52) or uses the 3′ splice acceptor (to produce Rep68 and Rep40). Consequently, four different functional Rep proteins with overlapping sequences can be synthesized from these promoters.

In the wild-type rep gene, the p40 promoter is located at the 3′ end. Transcription of the Cap proteins (VP1, VP2 and VP3) is initiated from this promoter in the wild-type AAV genome.

The four wild-type Rep proteins are Rep78, Rep68, Rep52 and Rep40. Hence the wild-type rep gene is one which encodes the four Rep proteins Rep78, Rep68, Rep52 and Rep40.

As used herein, the term “rep gene” includes wild-type rep genes and derivatives thereof; and artificial rep genes which have equivalent functions.

In one embodiment, the rep gene encodes functional Rep78, Rep68, Rep52 and Rep40 polypeptides. In another embodiment, the rep gene encodes functional Rep 78 and Rep 68 polypeptides. In some embodiments, the rep gene p19 promoter is non-functional.

The wild-type AAV (serotype 2) rep gene nucleotide sequence is given in SEQ ID NO: 13. In one embodiment, the term “rep gene” refers to a nucleotide sequence having at least 70%, 80%, 85%, 90%, 95%, 99% or 100% sequence identity to SEQ ID NO: 13 and which encodes one or more Rep78, Rep68, Rep52 and Rep40 polypeptides.

In some embodiments, the first nucleotide sequence encoding an AAV Rep polypeptide or a rep gene is not operably-associated with a functional promoter. In this way, a low level of expression of Rep polypeptides is obtained, wherein the expression level is sufficiently low such as not to prevent adenoviral growth and not to be sufficiently toxic to cells such as to prevent AAV production.

In the wild-type AAV, expression of the rep gene products are driven by the p5 and p19 promoters.

As used herein, the term “the rep gene is not operably-associated with a functional promoter” means that the rep gene does not comprise a functional p5 or a functional p19 promoter, and that the rep gene is not operably-associated with any other functional promoter, such that only baseline or minimal transcription of the rep gene is obtained.

In some preferred embodiments of the invention, the transcription of the rep gene will be driven by a polymerase II promoter. The promoter may be inducible or constitutive. If the promoter is inducible, the inducing agent (chemical or protein or both) may be preferably added at the same time that CARE-based amplification is induced. If the promoter is constitutive, then the strength of the promoter should not be too strong such that the rep gene is toxic to the cells when the CARE elements are not being amplified.

In some embodiments, the p5 promoter of one of the CARE elements may drive the rep gene. However, when used in such configuration, the cells should not also stably encode and express adenovirus E1A. The presence of the E1A protein is known to activate the p5 promoter and therefore cause toxicity to the cells. In preferred embodiments, the rep gene will not be driven by a p5 promoter inside a CARE element. As used herein, the term “cap gene” refers to a gene that encodes one or more open reading frames (ORFs), wherein each of said ORFs encodes an AAV Cap structural protein, or variant or derivative thereof. These AAV Cap structural proteins (or variants or derivatives thereof) form the AAV capsid.

The three Cap proteins must function to enable the production of an infectious AAV virus particle which is capable of infecting a suitable cell. The three Cap proteins are VP1, VP2 and VP3, which are generally 87 kDa, 72 kDa and 62 kDa in size, respectively. Hence the cap gene is one which encodes the three Cap proteins VP1, VP2 and VP3.

In the wild-type AAV, these three proteins are translated from the p40 promoter to form a single mRNA. After this mRNA is synthesized, either a long or a short intron can be excised, resulting in the formation of a 2.3 kb or a 2.6 kb mRNA.

The AAV capsid is composed of 60 capsid protein subunits (VP1, VP2, and VP3) that are arranged in an icosahedral symmetry in a ratio of 1:1:10, with an estimated size of 3.9 MDa.

As used herein, the term “cap gene” includes wild-type cap genes and derivatives thereof, and artificial cap genes which have equivalent functions. The AAV (serotype 2) cap gene nucleotide sequence and Cap polypeptide sequences are given in SEQ ID NOs: 14 and 15, respectively. As used herein, the term “cap gene” refers preferably to a nucleotide sequence having the sequence given in SEQ ID NO: 14 or a nucleotide sequence encoding SEQ ID NO: 15; or a nucleotide sequence having at least 70%, 80%, 85% 90%, 95% or 99% sequence identity to SEQ ID NO: 14 or at least 80%, 90%, 95% or 99% nucleotide sequence identity to a nucleotide sequence encoding SEQ ID NO:15, and which encodes VP1, VP2 and VP3 polypeptides.

The rep and cap genes are preferably viral genes or derived from viral genes. More preferably, they are AAV genes or derived from AAV genes. In some embodiments, the AAV is an Adeno-associated dependoparvovirus A. In other embodiments, the AAV is an Adeno-associated dependoparvovirus B.

11 different AAV serotypes are known. All of the known serotypes can infect cells from multiple diverse tissue types. Tissue specificity is determined by the capsid serotype. The AAV may be from serotype 1, 2, 3, 4, 5, 6, 7, 8, 9, 10 or 11. Preferably, the AAV is serotype 1, 2, 5, 6, 7, 8 or 9. Most preferably, the AAV serotype is 5 (i.e. AAV5).

The rep and cap genes (and each of the protein-encoding ORFs therein) may be from one or more different viruses (e.g. 2, 3 or 4 different viruses). For example, the rep gene may be from AAV2, whilst the cap gene may be from AAV5.

It is recognised by those in the art that the rep and cap genes of AAV vary by clade and isolate. The sequences of these genes from all such clades and isolates are encompassed herein, as well as derivatives thereof.

In other embodiments, the first nucleotide sequence does not comprise an AAV rep gene sequence or does not comprise an AAV cap gene sequence or does not comprise the sequence of an AAV Inverted Terminal Repeat (ITR). In other embodiments, the first nucleotide sequence does not comprise an AAV sequence. In some embodiments, the CARE element is not linked (contiguously or non-contiguously) to an AAV rep or cap gene.

In a preferred embodiment, the first nucleotide sequence comprises a recombinant AAV genome, preferably wherein the recombinant AAV genome comprises inverted terminal repeats flanking a transgene.

As used herein, the term “recombinant AAV genome” refers to an AAV genome comprising AAV inverted terminal repeats (ITRs) flanking a (non-wild-type) intervening sequence, preferably wherein the intervening sequence is more than 100 bp in length.

In particular, the intervening sequence may be a first nucleotide sequence as defined above, e.g. a nucleotide sequence that encodes: a gene of interest (GOI) or a transgene; a therapeutic polypeptide or fragment thereof; a CRISPR enzyme or sgRNA; a gene from a virus which is known to infect a mammal; one or more polypeptides known to induce an immune response in humans; a gene from a retrovirus or a rhabdovirus; genes required to make a viral packaging cell line that encodes genes that are required to assemble a gene therapy viral vector or encodes a gene therapy transfer vector; genes required to make a viral producer cell line that encodes all the genes and a transfer vector that are required to produce a gene therapy vector; or one or more genes for lentiviral or adenoviral vectors. Preferably, the recombinant AAV genome comprises a transgene.

The recombinant AAV genome preferably does not comprise an AAV rep gene or an AAV cap gene.

As used herein, the terms “recombinant AAV genome”, “AAV Transfer vector” and “Transfer Plasmid” are used interchangeably. They all refer to a vector comprising 5′- and 3′-viral (preferably AAV) inverted terminal repeats (ITRs), flanking an intervening nucleotide sequence, preferably a transgene.

The terms “operably-associated” and “operably-linked” are used herein interchangeably. Both terms refer to the association of polynucleotides on a single nucleic acid fragment so that the function of one polynucleotide affects the function of the other polynucleotide. The polynucleotides may be juxtaposed, adjacent or contiguously-linked; one may be upstream of the other. The terms imply a physical connection between the polynucleotides within a distance which allows the function of one polynucleotide to affect the function of the other polynucleotide. For example, a promoter is operably-linked with a coding polynucleotide or functional RNA when the promoter is capable of affecting the expression of that coding polynucleotide or functional RNA (i.e. that the coding polynucleotide or functional RNA is under the transcriptional control of the promoter). Coding polynucleotides in sense or antisense orientation can be operably-linked to regulatory polynucleotides.

The CARE elements and the first nucleotide sequence are operably-linked. As used herein, the term “operably-linked” in the context of the CARE elements and the first nucleotide sequence means that the CARE elements and the first nucleotide sequence are linked in a manner such that the CARE elements promote the amplification of the first nucleotide sequence in the presence of an adenovirus and AAV. This means that the CARE elements and the first nucleotide sequence are present in the same nucleic acid molecule, e.g. they are juxtaposed, adjacent or contiguously-linked, with the CARE elements flanking the first nucleotide sequence.

Preferably, the first nucleotide sequence comprises one or more transcriptional and/or translational control elements (e.g. an enhancer, promoter, terminator sequence, etc.).

The first nucleotide sequence may additionally comprise a Kozak sequence (translation start site).

CARE-based amplification has been most extensively studied using adenovirus (AV) as the inducer of amplification. In some embodiments of the invention, it is preferable to prevent the AV from producing more AV particles whilst inducing CARE amplification. This is because the AV particles may then contaminate any material (e.g. AAV proteins or vaccine particles) that are produced during CARE-based amplification. It is therefore preferable to prevent AV particle formation.

In some preferred embodiments, the first nucleotide sequence encodes a shRNA or a siRNA against an AV Late gene mRNA. In other embodiments of the invention, cells comprising nucleic acid molecules of the invention encode a shRNA or a siRNA against an AV Late gene mRNA.

Preferred AV Late gene mRNAs include Hexon, Penton, Fibre, Protein VI, Protein IIIa, Protein VIII, Protein IX, Protein Mu, Protein VII, Protein V, Protein IX and Iva2 mRNAs. Most preferably, the shRNA will target more than one AV Late gene. The most preferable targets for shRNAs against AV Late genes are those that are integral for AV icosahedron formation (e.g. Hexon). By targeting these genes, AV particle assembly is therefore reduced or prevented.

One or more other factors may be supplied, in cis or in trans, in order to promote the amplification of the first nucleotide sequence.

In particular, one or more of the following factors may be supplied, either in the form of polypeptides or genes encoding such polypeptides: (i) one or more adenovirus Early gene products; (ii) one or more adenovirus Late gene products; and/or (iii) one or more AAV gene products. Each of these gene products may be supplied as separate moieties or combinations of moieties or by the infection of the host cell with adenovirus or AAV, as desired.

Preferably, the adenovirus Early gene products are selected from adenoviral E1A, E1B, E2A, VA RNA and E4. Adenoviral E1A and E1B gene products are preferably supplied by the use of a nucleic acid molecule of the invention in a cell line in which E1A and E1B genes are present (e.g. integrated into the host cell genomes, as in HEK293, PerC6 or 911 cells). Preferably, the one or more adenovirus Early gene products includes E2A. Adenoviral VA RNA and E4 gene products may be supplied by infection of the cell line with adenovirus.

In embodiments of the invention which relate to the production of AAVs, one or more adenovirus Early gene products may be required in order to effect the packaging of the AAVs. These gene products are preferably present within the host cell in an adenoviral vector.

Preferably, the adenovirus Late gene products are selected from one or more of adenoviral L4 22K, L4 33K and L4 100K polypeptides. Adenoviral Late gene products, e.g. L4 22K, L4 33K and L4 100K, may be supplied on separate nucleic acid molecules or by infection of the cell line with adenovirus. Preferred L4 22K and L4 33K sequences are disclosed herein. Preferably, the AAV gene products are Rep78/68. AAV gene products may be supplied in AAVs.

In some embodiments, the nucleic acid molecule comprises only two CARE elements or fragments thereof. In other embodiments, the nucleic acid molecule does not comprise more than 5, 10 or 20 CARE elements or fragments thereof. In other embodiments, the nucleic acid molecule comprises only two CARE elements or fragments thereof within any one stretch of 1,000 or 5,000 or 10,000 nucleotides of the nucleic acid molecule. In other embodiments, the nucleic acid molecule does not comprise CARE elements in both forward and reverse orientations.

In some embodiments, the nucleic acid molecule of the invention is integrated into a host cell genome, e.g. into a human chromosome. For example, 1-100, 1-20 or 5-10 copies of the nucleic acid molecule of the invention may be integrated into a host cell genome, e.g. into a human chromosome. In some preferred embodiments, copies of the nucleic acid molecule of the invention may be integrated into a plurality (e.g. 2-10 or more) of different locations in the chromosomes of the host cell, wherein the nucleic acid molecules are not concatenated together.

In some embodiments, the nucleic acid molecule of the invention is integrated into human chromosome 19 at the AAVS1 site. Adeno-associated virus (AAV) is the only known eukaryotic virus capable of targeting human chromosome 19 for integration at a well-characterized site, i.e. the AAVS1 site. The site-specific integration of AAV is mediated by Rep68 and Rep78. The latter viral proteins bind to both the viral genome and to the AAVS1 site on chromosome 19 through RRSs located in the viral genome and in the AAVS1 site. The AAVS1 site has been mapped by in situ hybridisation to human chromosome 19 at 19q13.4-qter. (Giraud, C., E. Winocour, and K. 1. Berns. 1994. Site-specific integration by adeno-associated virus is directed by a cellular DNA sequence. Proc. Natl. Acad. Sci. USA 91:10039-10043).

In some embodiments, the nucleic acid molecule of the invention is present within an episome, such as a plasmid or vector. In some embodiments, the plasmid or vector comprises a plurality (e.g. 2, 3, 4, 5, 6, 7, 8, 9 or 10, or more) of nucleic acid molecules of the invention. These nucleic acid molecules may be contiguous or non-contiguous.

In some embodiments, the first nucleic acid molecules may be pre-concatenated prior to introduction into the cell and the concatenated DNA molecule (e.g. plasmid or vector) will become integrated into the chromosome of the cell. As used herein, the term “concatenated” refers to a molecule having or comprising the structure:
{-[CARE element]-[first nucleotide sequence]-}
   where n is 2 or more (e.g. 2 or 3-10, or more).

For example, the above molecule might have the structure [first CARE element]-[first nucleotide sequence]-[second CARE element]-[first nucleotide sequence](i.e. where n=2). Such structures form a further embodiment of the invention.

In other embodiments, the nucleic acid molecule may be designed in such a way that the nucleic acid molecule may self-concatenate, either prior to entering a cell or once inside a cell. Such self-concatenation should enable the formation of the nucleic acid of the invention. Such self-concatenation will rely on compatible cohesive ends of the DNA molecules such that they directionally assemble to form a string of nucleic acid molecules of the invention based on DNA base-paring that favours said self-concatenation.

For example, the molecule may have or comprise the structure:
X1-{-[CARE element]-[first nucleotide sequence]-}ₙ-X2
   wherein n is 1 or more (e.g. 1 or 2-10, or more) and wherein X1 and X2 are independently stretches of single-stranded DNA, and wherein the nucleotide sequences of X1 and X2 are complementary or partially-complementary to each other. Such structures form a further embodiment of the invention.

The nucleotide sequences of X1 and X2 are complementary or partially-complementary to each other such that these structures are capable of forming linear concatemers of nucleic acid molecules of the invention.

In some embodiments, a DNA molecule may be used to bridge the components of the nucleic acid molecule (e.g. CARE element and first nucleotide sequence) of the invention to aid self assembly in vivo. This will be achieved through such a bridge sharing homology to the CARE element and the first nucleic nucleotide sequence, and as such favour the assembly of nucleic acid molecules of the invention by homology-dependent repair inside a cell.

The invention also provides a host cell comprising a nucleic acid molecule of the invention. The host cells may be isolated cells, e.g. they are not situated in a living animal or mammal. Preferably, the host cell is a mammalian cell. Examples of mammalian cells include those from any organ or tissue from humans, mice, rats, hamsters, monkeys, rabbits, donkeys, horses, sheep, cows and apes. Preferably, the cells are human cells. The cells may be primary or immortalised cells.

Preferred cells include HEK-293, HEK 293T, HEK-293E, HEK-293 FT, HEK-293S, HEK-293SG, HEK-293 FTM, HEK-293SGGD, HEK-293A, MDCK, C127, A549, HeLa, CHO, mouse myeloma, PerC6, 911 and Vero cell lines. HEK-293 cells have been modified to contain the E1A and E1B proteins and this obviates the need for these proteins to be supplied on a Helper Plasmid or within an adenoviral vector used in the invention. Similarly, PerC6 and 911 cells contain a similar modification and can also be used. Most preferably, the human cells are HEK293, HEK293T, HEK293A, PerC6 or 911 cells. Other preferred cells include Hela, CHO and VERO cells.

Other preferred cells include HEK-293AD cells and suspension CHO-X cells.

In yet other embodiments, the host cell additionally comprises one or both of:
(c) an AAV Transfer Plasmid comprising a transgene flanked by ITRs; and
(d) an adenoviral Helper Plasmid for AAV production comprising one or more genes selected from E1A, E1B, E2A, E4 and VA RNA.

In some embodiments of the invention, the Helper Plasmid comprises an E2A gene. In other embodiments, the Helper Plasmid does not comprise an E2A gene. In the latter case, the omission of the E2A gene reduces considerably the amount of DNA which is needed in the Helper Plasmid. Such cells are commonly known as packaging cells.

In some embodiments of the invention, the host cell does not comprise an adenovirus or a herpesvirus. In some embodiments (e.g. wherein the first nucleotide sequence comprises a rep gene), the host cell does not comprise an E1A gene or an E1A gene product. In some embodiments (e.g. wherein the first nucleotide sequence comprises a rep gene), the host cell does not comprise an E1B gene or an E1B gene product. In some embodiments (e.g. wherein the first nucleotide sequence comprises a adenovirus gene), the host cell does not comprise an AAV Rep gene product.

In some preferred embodiments, the AAV cap gene is integrated into the host cell genome under the control of a promoter that is capable of being activated by a polypeptide (an activator) that is encoded within the adenoviral vector.

In yet a further embodiment of the invention, an adenoviral vector of the invention encodes a polypeptide which is capable of transcriptionally-activating a (remote) promoter, for example a promoter which is present in a host cell. Preferably, the promoter in the host cell is one which is operably-associated with (i.e. drives expression of) an AAV cap gene.

In some embodiments, the adenoviral vector encodes a polypeptide which is capable of transcriptionally-activating a promoter which is not present in that adenoviral vector.

Examples of such activators include the VP16 transcriptional activator from the herpes simplex virus and the trans-activator domain from the p53 protein. Such activators may be linked to DNA-binding domains such as those that bind to a cumate-binding site or a tetracycline-binding site in the cap gene promoter. This allows transcription of the cap gene only to be induced when the adenoviral vector is present within the host cell, thereby reducing the burden of expressing the AAV cap gene during adenovirus manufacture.

The nucleic acid molecules, plasmids and vectors, and host cells of the invention may be made by any suitable technique. Recombinant methods for the production of the nucleic acid molecules and packaging cells of the invention are well known in the art (e.g. “Molecular Cloning: A Laboratory Manual” (Fourth Edition), Green, M R and Sambrook, J., (updated 2014)).

The expression of the transgenes and other desired genes (e.g. rep and cap genes) from the nucleic acid molecules of the invention may be assayed in any suitable assay, including but not limited to western blot, northern blot, ELISA, RT-QPCR, and dot blot. The amplification of the nucleic acid of the invention may be determined by using a suitable detection method e.g. by assaying for the number of genome copies per ml by qPCR (as described the Examples herein) and comparing this to a reference loci in the chromosome of the cell. Ideally such loci will be at 1 or 2 copies per reference genome and can therefore be used to compare amplification against by way of reference to relative copy number.

In yet a further embodiment, the invention provides a process for producing a modified host cell, the process comprising:
(a) introducing a nucleic acid molecule of the invention into a host cell, to produce a modified host cell.

In some embodiments, the nucleic acid molecule is in the form of a vector or plasmid.

In some embodiments, process is carried out under conditions such that the nucleic acid molecule becomes integrated into the host cell genome.

In some embodiments, the host cell is one which expresses or is capable of expressing the AAV Rep polypeptide and/or AAV Cap polypeptide and/or an AAV genome. For example, the host cell may be one in which one or more nucleic acid molecules of the invention comprising first nucleotide sequences which encode the AAV Rep polypeptide and/or Cap polypeptide are stably integrated. The nucleotide sequences which encode Rep polypeptide and/or Cap polypeptide are preferably operably-associated with suitable regulatory elements, e.g. inducible or constitutive promoters.

For example, the host cell may be one which comprises one or more DNA plasmids or vectors comprising nucleic acid molecules of the invention wherein the first nucleotide sequence encodes the AAV Rep polypeptide and/or Cap polypeptide. The nucleotide sequences which encode Rep polypeptide and/or Cap polypeptide are preferably operably-associated with suitable regulatory elements, e.g. inducible or constitutive promoters. The host cell may be an AAV packaging cell or an AAV producer cell.

As used herein, the term “introducing” one or more plasmids or vectors or nucleic acid molecules into a cell includes transformation, and any form of electroporation, conjugation, infection, transduction or transfection, inter alia.

In yet a further embodiment, the invention provides a method of amplifying a first nucleotide sequence in a host cell, the method comprising the step:
(a) culturing a host cell which comprises:
(i) a first nucleic acid molecule comprising:
   (a) a first CARE element, wherein the first CARE element comprises or consists of an AAV p5 promoter;
   (b) a first nucleotide sequence; and
   (c) a second CARE element, wherein the second CARE element comprises or consists of an AAV p5 promoter;
wherein (a), (b) and (c) are operably-associated in this order (5′-3′) in the nucleic acid molecule and wherein the first and second CARE elements are both in the same 5′-3′ orientation;
and optionally additionally
(ii) one or more second nucleic acid molecules comprising one or more promoters operably-associated with one or more adenovirus Early genes or adenovirus Late genes,
under conditions such that AAV Rep and one or more adenovirus Early gene products and/or adenovirus Late gene products are present in the host cell, thus promoting the amplification of the first nucleotide sequence.

In some embodiments, the method may additionally comprise the step, prior to Step (a), of introducing the first nucleic acid molecule into the host cell.

Preferably, the adenovirus Early gene products are selected from adenoviral E1A, E1B, E2A, VA RNA and E4, most preferably E1A. Preferably, the adenovirus Late gene products are selected from one or more of adenoviral L4 22K, L4 33K and L4 100K polypeptides.

In some embodiments, the second nucleic acid molecules are present in the host cell in an adenovirus or adenoviral vector. Preferably, the adenovirus Early gene products are selected from adenoviral E1A, E1B, E2A, VA RNA and E4, most preferably E1A.

Preferably, the adenovirus Late gene products are selected from one or more of adenoviral L4 22K, L4 33K and L4 100K polypeptides.

In some preferred embodiments, the first nucleotide sequence comprises: (i) an AAV rep gene; (ii) an AAV cap gene; (iii) an AAV rep and an AAV cap gene; or (iv) an AAV cap gene and a transgene; wherein any of (i)-(iv) may optionally be flanked by AAV ITRs.

In some embodiments wherein the first nucleotide sequence does not comprise an AAV rep gene, a recombinant AAV comprising an AAV rep gene is preferably present in the cell or is introduced into the cell, e.g. via a plasmid or vector. In other embodiments wherein the first nucleotide sequence does not comprise an AAV rep gene, a recombinant AV vector comprising an AAV rep gene inserted into the E1 region of an E1/E3-deleted adenoviral vector is preferably present in the cell or is introduced into the cell. The rep gene may also be inserted into other positions in the AV genome such as the E3 region.

In some embodiments, the rep gene is present within an AV vector, wherein the rep gene is not operably-associated with a functional promoter.

In yet a further embodiment, the invention provides a process for producing recombinant viral particles comprising a transgene, the process comprising the steps:
(a) culturing a host cell comprising:
   (i) a Transfer Plasmid comprising 5′- and 3′-viral ITRs flanking a transgene;
   (ii) a nucleic acid molecule of the invention comprising viral rep and cap genes, flanked by first and second CARE elements, the nucleic acid molecule either being present in an episomal plasmid or vector within the host cell or being integrated into the host cell genome; and
   (iii) sufficient AV helper genes for promoting amplification of the rep and cap genes and for packaging the Transfer Plasmid, the helper genes either being present in an episomal Helper Plasmid within the host cell, in an adenoviral vector or being integrated into the host cell genome;
   under conditions such that viral rep and cap genes are amplified and viral particles are assembled by the host cell; and
(b) harvesting packaged viral particles from the host cells or from the culture medium.

Preferably, the virus is an AAV. Preferably, the host cell is a viral packaging cell.

If the nucleic acid molecule of the invention is in a plasmid or vector, the plasmid or vector may be introduced into the host cell before or after the introduction of the Transfer Plasmid. If the helper genes are in a Helper Plasmid or adenoviral vector, the Helper Plasmid or adenoviral vector may be introduced into the host cell before or after the introduction of the Transfer Plasmid. In the above context, the term “adenoviral vector” encompasses an adenovirus.

The AV helper genes are preferably selected from one or more of (adenoviral) E1A, E1B, E4 and VA genes. In some embodiments of the invention, the helper genes additionally include an E2A gene. In other embodiments, the helper genes do not include an E2A gene. The AV helper genes may also include AV Late genes.

Preferably, the harvested virus particles are subsequently isolated and/or purified.

The host cell is cultured (in an appropriate medium) under conditions such that the first and, when present, second nucleic acid molecules are expressed. Suitable culture conditions for host cells are well known in the art (e.g. “Molecular Cloning: A Laboratory Manual” (Fourth Edition), Green, M R and Sambrook, J., (updated 2014)). In some embodiments, the host cell will be present in a culture medium, preferably a liquid culture medium.

As used herein, the term “amplifying” refers to the production of a plurality of DNA molecules. The plurality of DNA molecules are likely to comprise molecules of different lengths. The DNA regions adjacent to the nucleic acid molecule of the invention when inserted into the chromosome of a cell might also be amplified at lower levels (around 10-fold lower) than the nucleic acid molecule of the invention.

Each of the DNA molecules in the plurality of DNA molecules will have a nucleotide sequence which comprises part of the nucleotide sequences of the first and second CARE elements or fragments thereof. Without being bound by theory, it is expected that the minimum amplified region will be between the respective Rep Binding Sites (RBSs) within the CARE elements flanking the first nucleotide sequence, such that parts of each of the CARE elements and the first nucleotide sequence are amplified. In some embodiments, the entire CARE element may be amplified. In embodiments wherein the invention is constructed of a repeating concatemer of nucleic acid molecules of the invention, DNA amplification may proceed over multiple nucleic acid molecules of the invention during the amplification process.

Each of the DNA molecules in the plurality of DNA molecules will also have a nucleotide sequence which comprises all or part of the first nucleotide sequence.

In some embodiments, nucleic acid molecules of the invention may be present in the cell in 1-100 copies, preferably in 1-20 copies, and most preferably in 5-10 copies. The plurality of (amplified) DNA molecules may consist of 50-1000 discrete DNA molecules or more in the cell. The level of amplification attained will depend on the sequence length of the first nucleotide sequence, its complexity and the number of initial nucleic acid molecule copies in the cell.

The plurality of amplified DNA molecules are double-stranded DNA molecules. Such molecules will be created through single-stranded DNA synthesis followed by annealing of said single-stranded DNA molecules to form double-stranded DNA molecules. The plurality of amplified DNA molecules are linear, extra-chromosomal DNA molecules.

In some embodiments, one or more methods or processes of the invention additionally comprise the step:
isolating and/or purifying the amplified DNA molecules and/or the gene products thereof.

For example, the amplified DNA products may purified by DNA purification using silica resin in the presence of ethanol. Gene products (e.g. polypeptides) of the amplified DNA products may purified by any method which is suitable for the purification of that particular product, e.g. affinity chromatography.

In some preferred embodiments, the first nucleotide sequence comprises AAV rep and AAV cap genes; the host cell comprises E1A and E1B genes integrated into the host cell genome (e.g. HEK 293 cells); and the rep gene is not expressed from the p5 promoter within the first CARE element.

In other preferred embodiments, the first nucleotide sequence comprises an AAV rep gene and optionally also an AAV cap gene; the host cell does not express an E1A gene (and optionally also not an E1B gene)s from the host cell's chromosomes; an E1A gene (and optionally also an E1B gene) is expressed within the host cell from an adenoviral vector; and the rep gene is optionally expressed from the p5 promoter within the first CARE element.

In other preferred embodiments, the first nucleotide sequence comprises a transgene (e.g. an antigen, VLP protein or therapeutic protein); and the host cell optionally comprises E1A and E1B genes integrated into the host cell genome (e.g. HEK 293 cells).

In other preferred embodiments, the first nucleotide sequence comprises AAV rep and AAV cap genes; the host cell comprises E1A and E1B genes integrated into the host cell genome, wherein the E1A and E1B genes are not expressed from the E1 promoter (e.g. the E1A and E1B genes are expressed from an inducible promoter); and the rep gene is not expressed from the p5 promoter within the first CARE element.

In these preferred embodiments, the first nucleotide sequence may optionally additionally encode a shRNA or a siRNA against an AV Late gene mRNA or an AV E2B gene. In these preferred embodiment, in cells comprising nucleic acid molecules of the invention, the cell may optionally encode a shRNA or a siRNA against an AV Late gene mRNA or an AV E2B gene.

WO2019/020992 discloses that transcription of the Late adenoviral genes can be regulated (e.g. inhibited) by the insertion of a repressor element into the Major Late Promoter. By “switching off” expression of the adenoviral Late genes, the cell's protein-manufacturing capabilities can be diverted toward the production of a desired recombinant protein or AAV particles.

The Applicants subsequently found, however, that inhibiting the Late adenoviral genes by repressing the Major Late Promoter in the manner described in WO2019/020992 had the undesirable effect of inhibiting CARE-dependent replication of the rep and cap genes if those genes were integrated into the host cell genome.

Preferably, the adenoviral vector comprises a repressible Major Late Promoter (MLP), more preferably wherein the MLP comprises one or more repressor elements which are capable of regulating or controlling transcription of the adenoviral late genes, and wherein one or more of the repressor elements are inserted downstream of the MLP TATA box.

Preferably, the nucleic acid molecule, AAV cap gene, and Transfer plasmid are:
(i) stably integrated into the host cell genome; or (ii) present in the host cell in an episomal plasmid or vector.

Preferred features of the process for producing viral (preferably AAV) particles include the following:
wherein the one or more repressor elements are inserted between the MLP TATA box and the +1 position of transcription.
wherein the repressor element is one which is capable of being bound by a repressor protein.
wherein a gene encoding a repressor protein which is capable of binding to the repressor element is encoded within the adenoviral genome.
wherein the repressor protein is transcribed under the control of the MLP.
wherein the repressor protein is the tetracycline repressor, the lactose repressor or the ecdysone repressor, preferably the tetracycline repressor (TetR).
wherein the repressor element is a tetracycline repressor binding site comprising or consisting of the sequence set forth in SEQ ID NO: 16.
wherein the nucleotide sequence of the MLP comprises or consists of the sequence set forth in SEQ ID NO: 17 or 18.
wherein the presence of the repressor element does not affect production of the adenoviral E2B protein.
wherein a transgene is inserted within one of the adenoviral early regions, preferably within the adenoviral E1 region instead of in a Transfer Plasmid.
wherein the transgene comprises a Tripartite Leader (TPL) in its 5′-UTR.
wherein the transgene encodes a therapeutic polypeptide.
wherein the transgene encodes a virus protein, preferably a protein that is capable of assembly in or outside of a cell to produce a virus-like particle, preferably wherein the transgene encodes Norovirus VP1 or Hepatitis B HBsAG.

It is particularly preferred that one or more of the repressor elements are inserted downstream of the MLP TATA box.

There are many established algorithms available to align two amino acid or nucleic acid sequences. Typically, one sequence acts as a reference sequence, to which test sequences may be compared. The sequence comparison algorithm calculates the percentage sequence identity for the test sequence(s) relative to the reference sequence, based on the designated program parameters. Alignment of amino acid or nucleic acid sequences for comparison may be conducted, for example, by computer-implemented algorithms (e.g. GAP, BESTFIT, FASTA or TFASTA), or BLAST and BLAST 2.0 algorithms.

Percentage amino acid sequence identities and nucleotide sequence identities may be obtained using the BLAST methods of alignment (Altschul et al. (1997), “Gapped BLAST and PSI-BLAST: a new generation of protein database search programs”, Nucleic Acids Res. 25:3389-3402; and http://www.ncbi.nlm.nih.gov/BLAST). Preferably the standard or default alignment parameters are used.

Standard protein-protein BLAST (blastp) may be used for finding similar sequences in protein databases. Like other BLAST programs, blastp is designed to find local regions of similarity. When sequence similarity spans the whole sequence, blastp will also report a global alignment, which is the preferred result for protein identification purposes. Preferably the standard or default alignment parameters are used. In some instances, the “low complexity filter” may be taken off.

BLAST protein searches may also be performed with the BLASTX program, score=50, wordlength=3. To obtain gapped alignments for comparison purposes, Gapped BLAST (in BLAST 2.0) can be utilized as described in Altschul et al. (1997) Nucleic Acids Res. 25: 3389. Alternatively, PSI-BLAST (in BLAST 2.0) can be used to perform an iterated search that detects distant relationships between molecules. (See Altschul et al. (1997) supra). When utilizing BLAST, Gapped BLAST, PSI-BLAST, the default parameters of the respective programs may be used.

With regard to nucleotide sequence comparisons, MEGABLAST, discontiguous-megablast, and blastn may be used to accomplish this goal. Preferably the standard or default alignment parameters are used. MEGABLAST is specifically designed to efficiently find long alignments between very similar sequences. Discontiguous MEGABLAST may be used to find nucleotide sequences which are similar, but not identical, to the nucleic acids of the invention.

The BLAST nucleotide algorithm finds similar sequences by breaking the query into short subsequences called words. The program identifies the exact matches to the query words first (word hits). The BLAST program then extends these word hits in multiple steps to generate the final gapped alignments. In some embodiments, the BLAST nucleotide searches can be performed with the BLASTN program, score=100, wordlength=12.

One of the important parameters governing the sensitivity of BLAST searches is the word size. The most important reason that blastn is more sensitive than MEGABLAST is that it uses a shorter default word size (11). Because of this, blastn is better than MEGABLAST at finding alignments to related nucleotide sequences from other organisms. The word size is adjustable in blastn and can be reduced from the default value to a minimum of 7 to increase search sensitivity.

A more sensitive search can be achieved by using the newly-introduced discontiguous megablast page (www.ncbi.nlm.nih.gov/Web/Newsitr/FallWinter02/blastlab.html). This page uses an algorithm which is similar to that reported by Ma et al. (Bioinformatics. 2002 March; 18(3): 440-5). Rather than requiring exact word matches as seeds for alignment extension, discontiguous megablast uses non-contiguous word within a longer window of template. In coding mode, the third base wobbling is taken into consideration by focusing on finding matches at the first and second codon positions while ignoring the mismatches in the third position. Searching in discontiguous MEGABLAST using the same word size is more sensitive and efficient than standard blastn using the same word size. Parameters unique for discontiguous megablast are: word size: 11 or 12; template: 16, 18, or 21; template type: coding (0), non-coding (1), or both (2).

In some embodiments, the BLASTP 2.5.0+ algorithm may be used (such as that available from the NCBI) using the default parameters. In other embodiments, a BLAST Global Alignment program may be used (such as that available from the NCBI) using a Needleman-Wunsch alignment of two protein sequences with the gap costs: Existence 11 and Extension 1.

As used herein, the term “sequence identity” in the context of amino acid sequences may alternatively be replaced by “sequence similarity”. The term “similarity” allows conservative substitutions of amino acid residues having similar physicochemical properties over a defined length of a given alignment. The percentage of similarity is determinable with any reasonable similarity-scoring matrix.

In yet other embodiments, there is provided a nucleic acid molecule comprising:
(a) a first CARE element or a fragment thereof;
(b) a first nucleotide sequence; and
(c) a second CARE element or a fragment thereof;
   wherein (a), (b) and (c) are operably-associated in this order in the nucleic acid molecule and wherein the first and second CARE elements, or fragments thereof, are both in the same 5′-3′ orientation.

In some embodiments, the first and/or second CARE element comprises:
(i) an AAV p5 promoter; and
(ii) a 5′ portion of the AAV rep gene.

In some embodiments, the first and/or second CARE element comprises:
(iii) a region of an AAV genome corresponding to nucleotides 146 to 190 of the AAV2 genome;
or a variant thereof having at least 80% (preferably at least 85%, 90%, 95% or 99%) sequence identity thereto, or a fragment thereof which is at least 50% (preferably at least 60%, 70%, 80%, 90% or 95%) of the length of the region.

The disclosure of each reference set forth herein is specifically incorporated herein by reference in its entirety.

### EXAMPLES

The present invention is further illustrated by the following Examples, in which parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### Example 1: Production of CARE-Driven Amplification Cassettes

An enhanced green fluorescent (eGFP) expression cassette was produced as a reporter cassette. This comprised a Kozak-Shine Dalgarno sequence and SV40 polyadenylation sequence (poly(A) signal) flanking an eGFP gene.

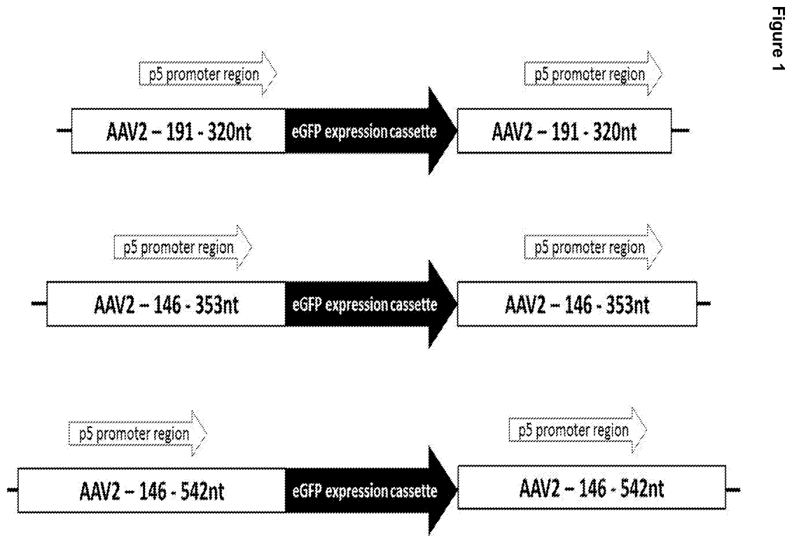

Fragments of the wild-type AAV2 sequence (NC_001401.2) were cloned up- and down-stream of the eGFP expression cassette, maintaining 5′ to 3′ directionality of all components, to produce three CARE-eGFP-CARE amplification cassettes, as shown in FIG. 1.

The first amplification cassette comprised AAV2 nucleotides 191-320 (“191-320 CARE”); the second amplification cassette comprised AAV2 nucleotides 146-353 (“146-353 CARE”); and the third amplification cassette comprised AAV2 nucleotides 146-542 (“146-542 CARE”). The third amplification cassette additionally comprised AAV rep genes. In this cassette, the AAV Rep open reading frame (initiating from position 321 nt) was mutated by substitution to remove ATG start codons.

The native AAV p5 promoter region was maintained in all instances, including TATA box, Rep binding site (RBS) and terminal resolution site (trs) to initiate transcription of the eGFP ORF. An “eGFP Control” cassette was also produced, with no AAV2 (CARE) nucleotide regions. A “CARE Control” cassette was also produced, which lacked the eGFP cassette.

### Example 2: CARE Directed DNA Amplification of Integrated CARE-eGFP-CARE Vector

CARE-eGFP-CARE amplification cassettes, as produced in Example 1, were cloned into integration plasmids encoding for puromycin resistance and the ϕC31 integrase attB recognition site (Thyagarajan et al. 2001. Mol. Cell. Biol. 21, 3926-3934). HeLa cells were seeded at 9.6e5 cells/well of a 6-well plate 16 hours before transfecting with ϕC31 expression plasmid and the desired integration construct at a ratio of 10:1 of the total 2.5 μg of DNA transfected. Cells were placed under 0.5 μg/mL puromycin selection and stable pooled cell lines grown out to T75 cm² flasks after 2-3 weeks.

To test for CARE-directed amplification, cell lines were seeded at 9.6e5 cells/well of a 6-well plate, without selection,16 hours before transfecting with 0.25 μg Rep78 expression plasmid and 2.25 μg stuffer DNA. The media was exchanged 24 hours post transfection. After a further 24 hours, Rep78 transfected cells were counted and seeded at 9.6e4 cells/well of a 48-well plate in sextuplicate.

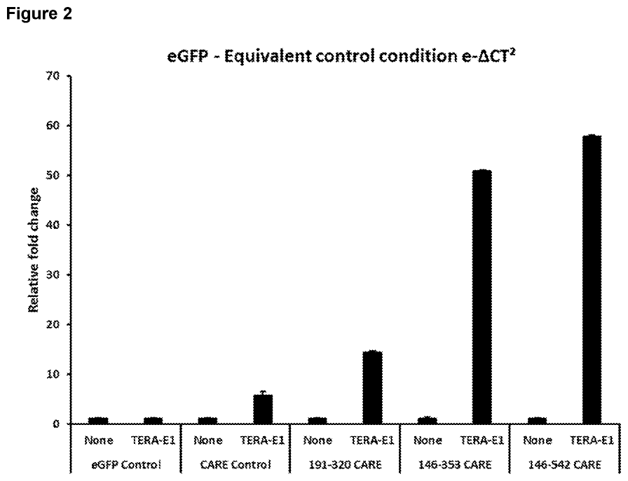

The following day, three wells from each cell line were inoculated with TERA-E1 (see Example 3) at a multiplicity of infectivity of 50 in the presence of 0.5 μg/mL doxycycline. The remaining three non-infected wells (labelled “None” in FIG. 2) were treated with the equivalent volume of DMSO only. Genomic DNA was harvested from each well 96 hours post-infection and quantified by qPCR in triplicate, 10 ng DNA per reaction. SYBR-based qPCR was performed against eGFP and the internal host genes RPPH1 and TERT. Relative eGFP values were calculated for each sample by geometric normalisation of the internal host genes (Vandesompele et al. 2002. Genome Biol. 2002 Jun. 18; 3(7):RESEARCH0034). Relative fold change to the non-infected samples is shown in FIG. 2 for each cell line.

“eGFP Control” and “CARE Control” represent HeLa cell lines generated in the same manner as the CARE-eGFP-CARE cell lines except lacking any AAV sequences or the eGFP cassette respectively.

These results demonstrate that significant levels of amplification of the eGFP cassette occurred in the amplification cassettes which comprised eGFP genes flanked by CARE elements.

### Example 3: siRNA Knockdown of Ad5 E2B Transcription Unit Decrease Adenovirus Genome Replication

WO2019/020992 discloses that transcription of the Late adenoviral genes can be regulated (e.g. inhibited) by the insertion of a repressor element into the Major Late Promoter (MLP). By “switching off” expression of the adenoviral Late genes, the cell's protein-manufacturing capabilities can be diverted toward the production of a desired recombinant protein or AAV particles.

MLP-repressible adenoviruses (“TERA-E1”) wherein the E3 region was deleted were produced in accordance with the method disclosed in WO2019/020992.

Ad5-E1 (a serotype 5 adenovirus, wherein the virus E3 region was deleted) was generated by molecular cloning methods and produced from HEK293 cells.

HeLaRC32 cells were seeded in 48-well tissue culture plates at 9.0e4 cells/well and cells were transfected with siRNA targeting Ad5 E2B unit (Kneidinger et al. 2012. Antiviral Res, 94: 195-207), scrambled siRNA (NC) or mock transfection. 24-hours after transfection, cells were infected with TERA-E1 or Ad5-E1 at an MOI of 50 and transfected with a plasmid encoding the AAV transfer genome (pAAV-EGFP). Cells were treated with doxycycline 0.5 μg/mL or DMSO at 4 hours after infection. Total DNA was extracted at 72 hours post-infection and quantified by qPCR using primers and probe against Ad5 hexon.

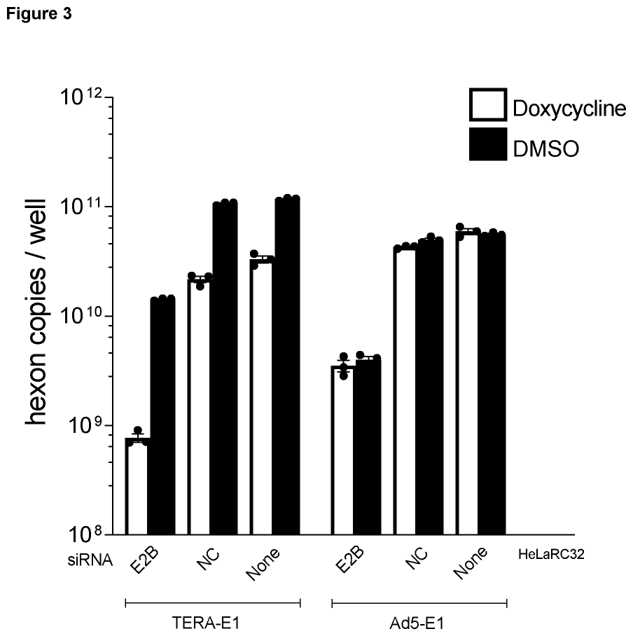

The results are given in FIG. 3, which show that siRNA targeting Ad5 E2B genes decreases replication of TERA-E1 and Ad5-E1 genomes in HeLaRC32 cells. The same principle may be applied to the production of AAVs in a process of the invention in order to suppress the production of AV particles.

### Example 4: siRNA Knockdown of Ad5 E2B Transcription Unit Increase Amplification of AAV Rep DNA from Stable AAV Packaging HeLaRC32 Cell

TERA E1 and Ad5 E1 recombinant adenoviruses were produced as described in Example 3.

HeLaRC32 cells were seeded in 48-well tissue culture plates at 9.0e4 cells/well and cells were transfected with siRNA targeting Ad5 E2B unit (Kneidinger et al. 2012. Antiviral Res, 94: 195-207), scrambled siRNA (NC) or mock transfection. 24-hours after transfection, cells were infected with TERA-E1 or Ad5-E1 at an MOI of 50 and transfected with a plasmid encoding the AAV transfer genome (pAAV-EGFP). Cells were treated with doxycycline 0.5 μg/mL or DMSO at 4 hours after infection. Total DNA was extracted at 72 hours post-infection and quantified by qPCR using primers and a probe against the AAV2 rep gene.

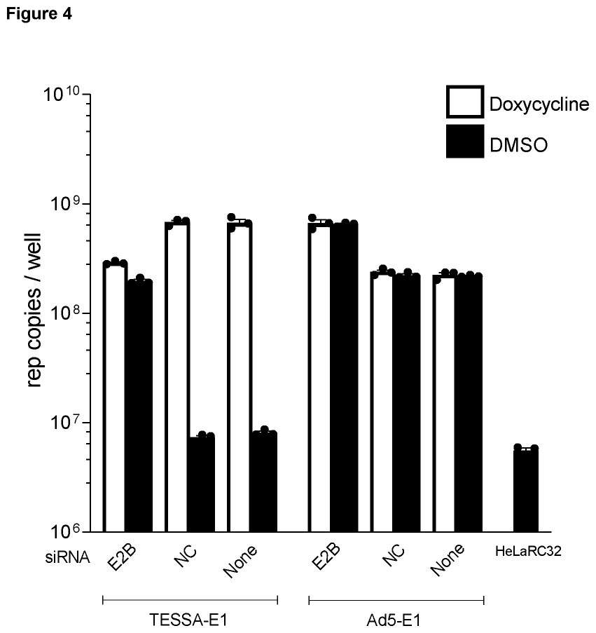

The results are given in FIG. 4, which show that siRNA targeting the Ad5 E2B gene increases DNA amplification of AAV2 rep from HeLaRC32 cells infected with Ad5-E1 and also TERA-E1 during MLP-repression (DMSO group). The same principle may be applied to the production of AAVs in a process of the invention in order to enhance the production of the AAV rep gene.

### Example 5: siRNA Knockdown of Ad5 E2B Transcription Unit Increase Amplification of AAV Cap DNA from Stable AAV Packaging HeLaRC32 Cell

TERA E1 and Ad5 E1 recombinant adenoviruses were produced as described in Example 3.

HeLaRC32 cells were seeded in 48-well tissue culture plates at 9.0e4 cells/well and cells were transfected with siRNA targeting Ad5 E2B unit (Kneidinger et al. 2012. Antiviral Res, 94: 195-207), scrambled siRNA (NC) or mock transfection. 24-hours after transfection, cells were infected with TERA-E1 or Ad5-E1 at an MOI of 50 and transfected with plasmid encoding the AAV transfer genome (pAAV-EGFP). Cells were treated with doxycycline 0.5 μg/mL or DMSO at 4 hours after infection. Total DNA was extracted at 72 hours post-infection and quantified by qPCR using primers and probe against AAV2 cap.

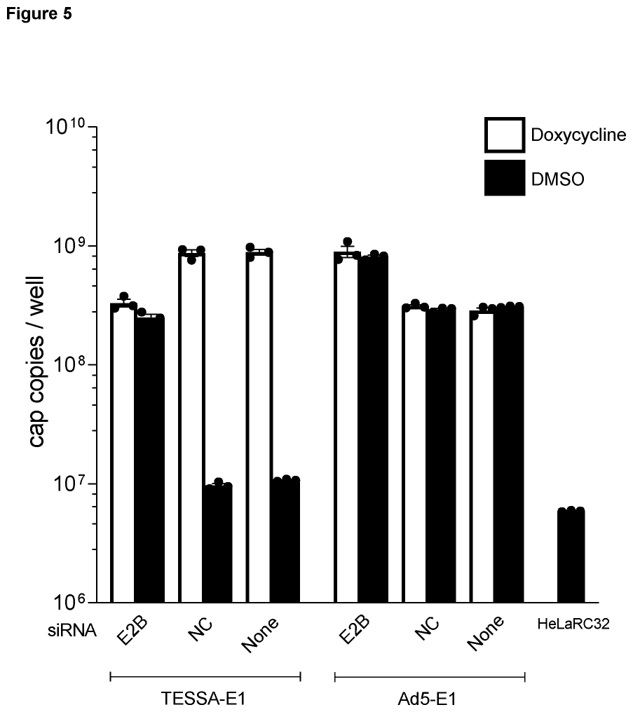

The results are given in FIG. 5, which show that siRNA targeting Ad5 E2B unit increases DNA amplification of AAV2 cap from HeLaRC32 cells infected with Ad5-E1 and also TERA-E1 during MLP-repression (DMSO group). The same principle may be applied to the production of AAVs in a process of the invention in order to enhance the production of the AAV cap gene.

### Example 6: CARE-Directed DNA Amplification of Integrated CARE-eGFP-CARE Vector by Transfection of Rep78 in 293AD Cell Lines

CARE-eGFP-CARE amplification cassettes were cloned into integration plasmids encoding puromycin resistance and the 0C31 integrase attB recognition site (Thyagarajan et al. 2001. Mol. Cell. Biol. 21, 3926-3934). 293AD cells were seeded at 9.6e5 cells/well of a 6-well plate 16 hours before transfecting with 0C31 expression plasmid and the desired integration construct at a ratio of 10:1 of the total 2.5 μg of DNA transfected. Cells were placed under 0.5 μg/mL puromycin selection and stable pooled cell lines were grown out to T75 cm² flasks after 2-3 weeks.

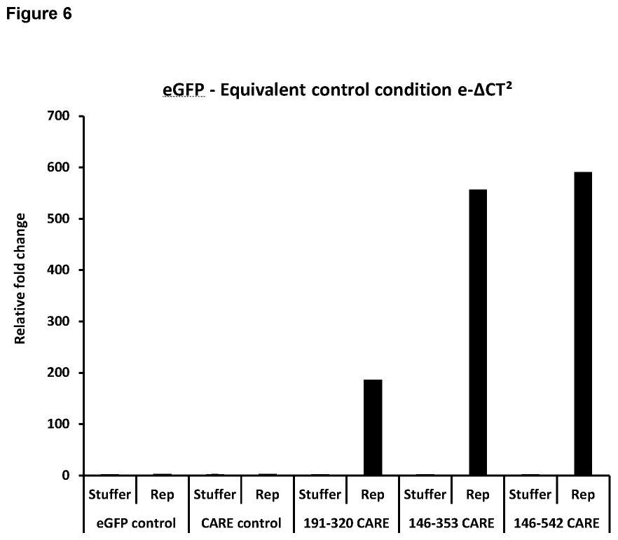

To test for CARE-directed amplification, cell lines were seeded at 9.6e5 cells/well of a 6-well plate, without selection,16 hours before transfecting with 0.25 μg Rep78 expression plasmid and 2.25 ug stuffer DNA (labelled “Rep” in FIG. 6) or 2.5 μg stuffer DNA only (labelled “Stuffer” in FIG. 6). The media was exchanged 24 hours post-transfection. After a further 24 hours, transfected cells were counted and seeded at 9.6e4 cells/well of a 48-well plate in triplicate. Genomic DNA was harvested from each well 144 hours post-transfection and quantified by qPCR in triplicate, 10 ng DNA per reaction. SYBR-based qPCR was performed against eGFP and the internal host genes RPPH1 and TERT. Relative eGFP values were calculated for each sample by geometric normalisation of the internal host genes (Vandesompele et al. 2002. Genome Biol. 2002 Jun. 18; 3(7):RESEARCH0034). Relative fold change to the stuffer DNA transfection samples is shown for each cell line in FIG. 6.

“eGFP Control” and “CARE control” represent 293AD cell lines generated in the same manner as the CARE-eGFP-CARE cell lines except lacking any AAV sequences or the eGFP cassette respectively.

The results in FIG. 6 show that the AAV Rep78 protein alone is sufficient to induce significant CARE-directed amplification in 293AD cells lines which natively encode and express the Adenovirus E1A and E1B genes.

### Example 7: CARE-Directed DNA Amplification of Integrated CARE-eGFP-CARE Vector in 293AD Cell Lines

CARE-eGFP-CARE amplification cassettes were cloned into integration plasmids encoding puromycin resistance and the ϕC31 integrase attB recognition site (Thyagarajan et al. 2001. Mol. Cell. Biol. 21, 3926-3934). 293AD cells were seeded at 9.6e5 cells/well of a 6-well plate 16 hours before transfecting with ϕC31 expression plasmid and the desired integration construct at a ratio of 10:1 of the total 2.5 μg of DNA transfected. Cells were placed under 0.5 μg/mL puromycin selection and stable pooled cell lines were grown out to T75 cm² flasks after 2-3 weeks.

To test for CARE-directed amplification, cell lines were seeded at 9.6e5 cells/well of a 6-well plate, without selection,16 hours before transfecting with 0.25 μg Rep78 expression plasmid and 2.25 ug stuffer DNA or 2.5 μg stuffer DNA only. The media was exchanged 24 hours post-transfection. After a further 24 hours, transfected cells were counted and seeded at 9.6e4 cells/well of a 48-well plate in sextuplicate.

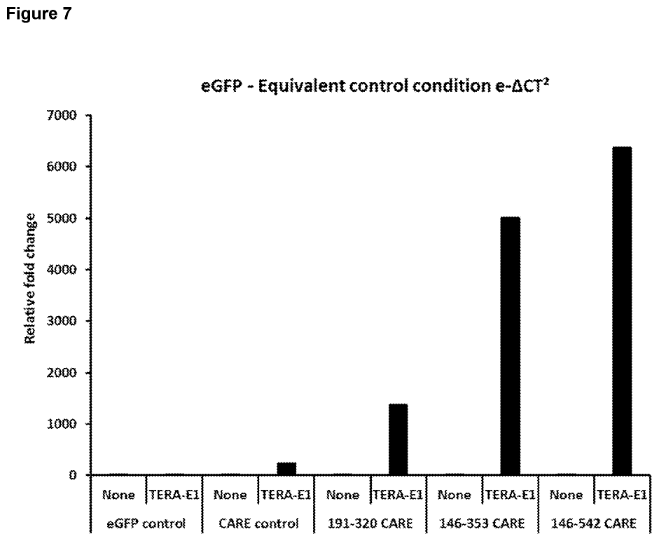

The following day, three wells from each cell line were inoculated with TERA-E1 at a multiplicity of infectivity of 50 in the presence of 0.5 μg/mL doxycycline. The remaining three non-infected wells (labelled “None” in FIG. 7) were treated with the equivalent volume of DMSO only.

Genomic DNA was harvested from each well 96 hours post infection and quantified by qPCR in triplicate, 10 ng DNA per reaction. SYBR-based qPCR was performed against eGFP and the internal host genes RPPH1 and TERT. Relative eGFP values were calculated for each sample by geometric normalisation of the internal host genes (Vandesompele et al. 2002. Genome Biol. 2002 Jun. 18; 3(7):RESEARCH0034). Relative fold change to the stuffer only transfected, non-infected samples is shown for each cell line in FIG. 7.

“eGFP Control” and “CARE control” represent 293AD cell lines generated in the same manner as the CARE-eGFP-CARE cell lines except lacking any AAV sequences or the eGFP cassette respectively.

The results in FIG. 7 show that 293 cell lines achieve greater levels of CARE-directed amplification of the eGFP cassette in the presence of Rep78 and Adenovirus helper function than Rep78.

### Example 8: CARE Directed DNA Amplification of Integrated CARE-eGFP-CARE Vector in Suspension CHO-X Cells

CARE-eGFP-CARE amplification cassettes were cloned into integration plasmids encoding puromycin resistance and the (VC31 integrase attB recognition site (Thyagarajan et al. 2001. Mol. Cell. Biol. 21, 3926-3934). Suspension CHO-X cells were seeded at 3e6 cells/well of a 24 deep-well plate 16 hours before transfecting with ϕC31 expression plasmid and the desired integration construct at a ratio of 10:1 of the total 4.5 μg of DNA transfected. Cells were placed under 5.0 μg/mL puromycin selection and stable pooled cell lines grown out to E125 flasks after 2-3 weeks.

To test for CARE-directed amplification, cell lines were seeded at 3e6 cells/well of a 24 deep-well plate, without selection,16 hours before transfecting with 0.45 μg Rep78 expression plasmid and 4.05 μg stuffer DNA or 4.5 μg stuffer DNA only. After a further 48 hours, transfected cells were counted and seeded at 6e5 cells/well of a 24 deep-well plate in duplicate.

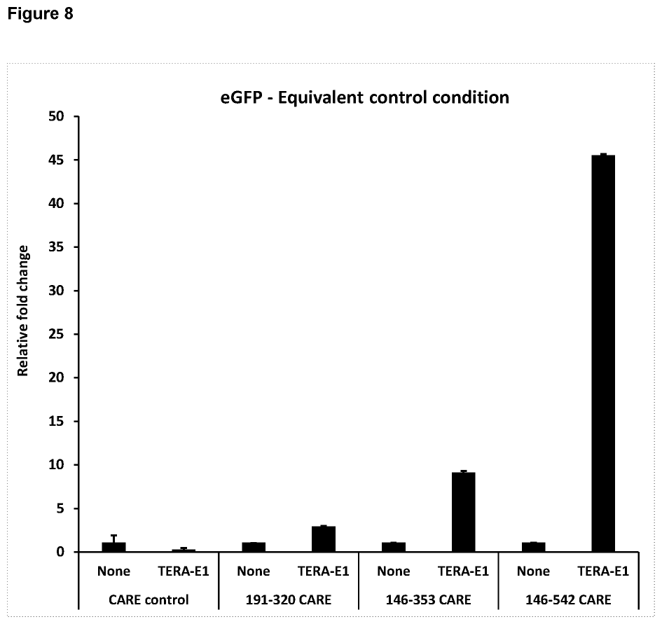

The following day, cells were either inoculated with TERA-E1 at a multiplicity of infectivity of 10 in the presence of 0.5 ug/mL doxycycline or mock-infected by only treating with the equivalent volume of DMSO. Genomic DNA was harvested from each well 72 hours post-infection and quantified by qPCR in triplicate, 10 ng DNA per reaction. SYBR-based qPCR was performed against eGFP. Relative fold change to the stuffer only transfected, non-infected samples is shown for each cell line in FIG. 8. CARE control represents a CHO-X cell line generated in the same manner as the CARE-eGFP-CARE cell lines except lacking the eGFP cassette.

The results in FIG. 8 show that CARE-directed amplification is achievable in Chinese hamster ovary (CHO) cells, a non-human derived cell line widely used for large-scale industrial therapeutic protein production.

### Example 9: CARE Directed DNA Amplification of Integrated CARE-GOI-CARE Vector in 293AD Cell Lines

Various genes of interest (GOIs) were cloned into CARE-GOI-CARE integration plasmids encoding puromycin resistance and the ϕC31 integrase attB recognition site (Thyagarajan et al. 2001. Mol. Cell. Biol. 21, 3926-3934). Each GOI reporter cassette comprised a heterologous promoter upstream of a Kozak-Shine Dalgarno sequence, the GOI and a SV40 polyadenylation sequence (poly(A) signal), except in the case of the luciferase cassette which lacked the heterologous promoter. Each reporter cassette was flanked by CARE elements maintaining the 5′ to 3′ directionality.

293AD cells were seeded at 9.6e5 cells/well of a 6-well plate 16 hours before transfecting with ϕC31 expression plasmid and the desired integration construct at a ratio of 10:1 of the total 2.5 μg of DNA transfected. Cells were placed under 0.5 μg/mL puromycin selection and stable pooled cell lines grown out to T75 cm² flask after 2-3 weeks.

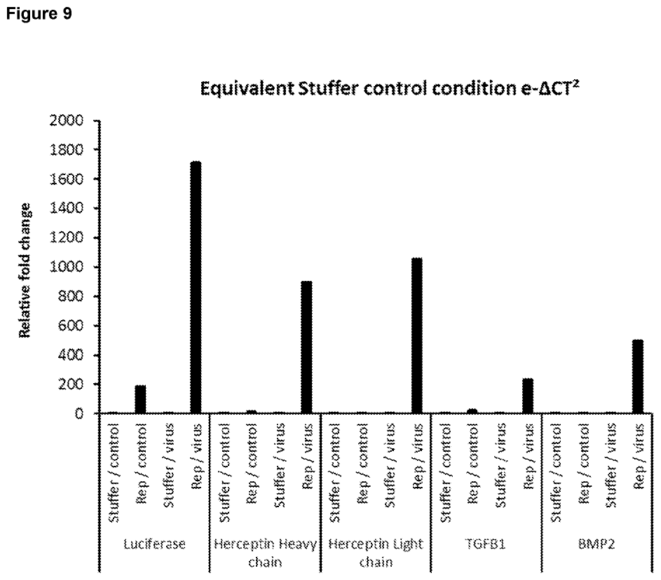

To test for CARE-directed amplification, cell lines were seeded at 9.6e5 cells/well of a 6-well plate, without selection,16 hours before transfecting with 0.25 μg Rep78 expression plasmid and 2.25 ug stuffer DNA (labelled as “Rep/” in FIG. 9) or 2.5 μg stuffer DNA only (labelled as “Stuffer/” in FIG. 9). The media was exchanged 24 hours post-transfection. After a further 24 hours, transfected cells were counted and seeded at 8.4e5 cells/well of a 6-well plate in duplicate.

The following day, one well from each transfection was inoculated with TERA-E1 (labelled as “/virus” in FIG. 9) at a multiplicity of infectivity of 10 in the presence of 0.5 μg/mL doxycycline. The remaining non-infected well was treated with the equivalent volume of DMSO only (labelled as “/control” in FIG. 9). Genomic DNA was harvested from each well 96 hours post-infection and quantified by qPCR in triplicate, 10 ng DNA per reaction. SYBR-based qPCR was performed against eGFP and the internal host genes RPPH1 and TERT. Relative eGFP values were calculated for each sample by geometric normalisation of the internal host genes (Vandesompele et al. 2002. Genome Biol. 2002 Jun. 18; 3(7):RESEARCH0034). Relative fold change to the stuffer only transfected, non-infected samples (labelled as “Stuffer/control” in FIG. 9 is shown for each cell line in FIG. 9.

The results in FIG. 9 demonstrate the utility of CARE-directed amplification for different GOIs cassettes, including cassettes containing heterologous promoter sequences.

### Example 10: Further Identification of Important CARE Regions

A number of additional CARE-reporter gene-CARE plasmids were made with a view to identifying the regions of the CARE elements which were responsible for high levels of DNA amplification.

Each of the plasmids in the following table contained a first CARE element consisting of the specified “5′ AAV seq” sequence, an eGFP expression cassette, and a second CARE element consisting of the specified “3′ AAV seq” (sequence), similar to those shown in FIG. 1. CARE-directed amplification was assayed in a similar manner to that described in Example 2.

The results show that the pre-p5 promoter sequence (AAV2 nt 146-191) plays a significant role in the level of DNA amplification achieved, and that the sequence of the second CARE element is more important than that of the first CARE element. Furthermore, the results show that no rep-encoding sequence is required for CARE-based DNA amplification.

| | | | Relative fold | Relative fold |
|---|---|---|---|---|
| Plasmid | | | change-Rep | change- |
| id | 5′ AAV seq | 3′ AAV seq | only | Rep + Virus |
| R2909 | 191-353 | 146-353 | +265 | +6570 |
| R2901 | 146-320 | 146-353 | +191 | +6214 |
| R2899 | 146-320 | 146-320 | +171 | +5650 |
| R2903 | 146-353 | 146-320 | +175 | +5613 |
| R2911 | 191-320 | 146-353 | +148 | +5213 |
| R2005 | 146-542 | 146-542 | +150 | +3915 |
| R2895 | 191-353 | 191-353 | +125 | +2777 |
| R2897 | 146-353 | 191-353 | +134 | +2552 |
| R2907 | 146-353 | 191-320 | +123 | +2439 |
| R2003 | 191-320 | 191-320 | +112 | +1936 |
| R2008 | eGFP cassette control | | +1 | +2 |
| R2272 | CARE control | | * | * |
| R2009 | 146-353 | 146-353 | * | * |

The Sequence Listing attached to this patent application forms part of the description of this patent application.

### DESCRIPTION OF ADDITIONAL SEQUENCES

SEQ ID NO: 13 Rep nucleotide sequence (AAV serotype 2)
SEQ ID NO: 14 Cap nucleotide sequence (AAV serotype 2)
SEQ ID NO: 15 Cap amino acid sequence (AAV serotype 2)
SEQ ID NO: 16 TetR binding site
SEQ ID NO: 17 Modified MLP
SEQ ID NO: 18 Modified MLP
SEQ ID NO: 19 L4 22K Protein name: Human adenovirus D serotype 9 (HAdV-9) (UniProtKB—Q5TJ00)
SEQ ID NO: 20 Ad5 L4 22K
SEQ ID NO: 21 Ad5 L4 22K
SEQ ID NO: 22 Ad5 L4 33K
SEQ ID NO: 23 Ad5 L4 33K UniProtKB—P24940 (SF33K_ADE05)

### SEQUENCE LISTING FREE TEXT

<210> 16 <223> TetR binding site
<210> 17 <223> Modified MLP
<210> 18 <223> Modified MLP

1. A method of amplifying a first nucleotide sequence in a host cell, the method comprising the step:
(a) culturing a host cell which comprises:
(i) a first nucleic acid molecule comprising:
(a) a first CARE element, wherein the first CARE element comprises or consists of an AAV p5 promoter;
(b) a first nucleotide sequence; and
(c) a second CARE element, wherein the second CARE element comprises or consists of an AAV p5 promoter;
wherein (a), (b) and (c) are operably-associated in this order (5′-3′) in the nucleic acid molecule and wherein the first and second CARE elements are both in the same 5′-3′ orientation;
and optionally additionally
(ii) one or more second nucleic acid molecules comprising one or more promoters operably-associated with one or more adenovirus Early genes or adenovirus Late genes,
under conditions such that AAV Rep and one or more adenovirus Early gene products and/or adenovirus Late gene products are present in the host cell, thus promoting the amplification of the first nucleotide sequence.

2. The method as claimed in claim 1, wherein the method additionally comprises the step, prior to Step (a), of introducing the first nucleic acid molecule into the host cell.

3. The method as claimed in claim 1, wherein each AAV p5 promoter independently comprises:
(a) a TATA box,
(b) an AAV Rep binding site, and
(c) a trs-like element.

4. The method as claimed in claim 1, wherein each AAV p5 promoter independently comprises one or more of:
(d) a MLTF/USP1 binding site,
(e) a YY1-60 binding site, and
(f) a YY1+1 binding site.

5. The method as claimed in claim 1, wherein the first and/or second CARE element independently comprise or consist of:
(i) the region of an AAV genome corresponding to nucleotides 191-320, 191-353, or 191-542 of the AAV2 genome;
(ii) a molecule having the nucleotide sequence of one of SEQ ID NOs: 7, 9 or 10; or
(iii) a variant of (i) or (ii) having at least 80% or at least 90% sequence identify to (i) or (ii), or a fragment of (i) or (ii) having at least 80% or at least 90% of the length of (i) or (ii), wherein the variant or fragment is, in the presence of adenovirus and AAV Rep proteins, capable of promoting the amplification of a first nucleotide sequence which is flanked by two such variants or fragments, and wherein the variants or fragments are both in the same 5′-3′ orientation.

6. The method as claimed in claim 1, wherein the first and/or second CARE element independently comprise or consist of a pre-AAV p5 promoter region and an AAV p5 promoter.

7. The method as claimed in claim 6, wherein each nucleotide sequence of the pre-AAV p5 promoter region independently comprises or consists of:
(i) the region of an AAV genome corresponding to nucleotides 146-190 of the AAV2 genome,
(ii) a molecule having the nucleotide sequence of SEQ ID NO: 8; or
(iii) a variant of (i) or (ii) having at least 80% or at least 90% sequence identify to (i) or (ii), or a fragment of (i) or (ii) having at least 80% or at least 90% of the length of (i) or (ii).

8. The method as claimed in claim 1, wherein the first and/or second CARE element independently comprises or consists of:
(i) the region of an AAV genome corresponding to nucleotides 146-320, 146-353, or 146-542 of the AAV2 genome;
(ii) a molecule having the nucleotide sequence of one of SEQ ID NOs: 6, 11 or 12; or
(iii) a variant of (i) or (ii) having at least 80% or 90% sequence identify to (i) or (ii), or a fragment of (i) or (ii) having at least 80% or 90% of the length of (i) or (ii), wherein the variant or fragment is, in the presence of adenovirus and AAV Rep proteins, capable of promoting the amplification of a first nucleotide sequence which is flanked by two such variants or fragments, and wherein the variants or fragments are both in the same 5′-3′ orientation.

9. The method as claimed in claim 1, wherein the first and/or second CARE element independently additionally comprise a 5′ portion of the AAV rep gene.

10. The method as claimed in claim 1, wherein the first nucleotide sequence encodes:
(a) a therapeutic polypeptide;
(b) a CRISPR enzyme or sgRNA; or
(c) a viral gene.

11. The method as claimed in claim 1, wherein the first nucleotide sequence encodes a recombinant AAV genome.

12. The method as claimed in claim 1, wherein the first nucleotide sequence comprises:
(i) an AAV rep gene;
(ii) an AAV cap gene:
(iii) an AAV rep and an AAV cap gene; or
(iv) an AAV cap gene and a transgene;
wherein any of (i)-(iv) may optionally be flanked by AAV ITRs.

13. The method as claimed in claim 10, wherein the first nucleotide sequence additionally encodes a shRNA or a siRNA against an adenovirus Late gene mRNA.

14. The method as claimed in claim 12, wherein the host cell is one which expresses the adenovirus E1A protein, wherein the first nucleotide sequence comprises an AAV rep gene, and optionally an AAV cap gene, and wherein:
(i) the p5 promoter in the first CARE element is modified to prevent rep gene expression from the p5 promoter in the first CARE element; or
(ii) the rep gene is operably-associated with a heterologous inducible or repressible promoter.

15. The method as claimed in claim 12, wherein adenovirus E1A and/or E1B protein is not expressed from the host cell's chromosomes, wherein the adenovirus E1A and/or E1B protein is expressed in the host cell from an adenoviral vector, wherein the first nucleotide sequence comprises an AAV rep gene, and optionally an AAV cap gene and/or recombinant AAV genome, and wherein:
(i) the p5 promoter is capable of promoting expression of the rep gene; or
(ii) the p5 promoter is not capable of promoting expression of the rep gene.

16. The method as claimed in claim 1, wherein:
(i) the first nucleic acid molecule does not encode a functional Rep protein; and/or
(ii) the first nucleic acid molecule does not encode a functional Cap protein.

17. (canceled)

18. (canceled)

19. A process for producing recombinant viral particles comprising a transgene,
the process comprising the steps:
(a) culturing a host cell comprising:
(i) a Transfer Plasmid comprising 5′- and 3′-viral ITRs flanking a transgene;
(ii) a first nucleic acid molecule as defined in claim 1,
the first nucleotide sequence comprising viral rep and cap genes, the first nucleic acid molecule either being present in an episomal plasmid or vector within the host cell or being integrated into the host cell genome; and
(iii) sufficient AV helper genes for promoting amplification of the rep and cap genes and for packaging the Transfer Plasmid, the helper genes either being present in an episomal Helper Plasmid within the host cell, in an adenoviral vector or being integrated into the host cell genome;
under conditions such that viral rep and cap genes are amplified and viral particles are assembled by the host cell; and
(b) harvesting packaged viral particles from the host cells or from the culture medium.

20. (canceled)

21. A nucleic acid molecule comprising:
(a) a first CARE element, wherein the first CARE element comprises an AAV p5 promoter;
(b) a first nucleotide sequence; and
(c) a second CARE element, wherein the second CARE element comprises an AAV p5 promoter;
wherein one or both of the first and/or second CARE element independently additionally comprise a pre-AAV p5 promoter region, wherein (a), (b) and (c) are operably-associated in this order (5′-3′) in the nucleic acid molecule and wherein the first and second CARE elements are both in the same 5′-3′ orientation.

22. (canceled)

23. (canceled)

24. (canceled)

25. (canceled)

26. (canceled)

27. (canceled)

28. A host cell comprising a nucleic acid molecule as claimed in claim 21.

29. A process for producing a modified host cell, the process comprising:
(a) introducing a nucleic acid molecule as claimed in claim 21 into a host cell,
thereby producing a modified host cell.


SEQUENCE LISTING (25 sequences)

SEQ ID NO: 1 - DNA, 130 bp, adeno-associated virus 2
gtcctgtattagaggtcacgtgagtgttttgcgacattttgcgacaccatgtggtcacgctgggtatttaagcccgagtgagcacgcagggtctccattttgaagcgggaggtttgaacgcgcagccgcc

SEQ ID NO: 2 - DNA, 14 bp, adeno-associated virus
aggtcacgtgagtg

SEQ ID NO: 3 - DNA, 16 bp, adeno-associated virus
gcccgagtgagcacgc

SEQ ID NO: 4 - DNA, 16 bp, adeno-associated virus
gagcgagcgagcgcgc

SEQ ID NO: 5 - DNA, 16 bp, adeno-associated virus
cagcgagcgagcgagc

SEQ ID NO: 6 - DNA, 175 bp, adeno-associated virus 2
ggaggggtggagtcgtgacgtgaattacgtcatagggttagggaggtcctgtattagaggtcacgtgagtgttttgcgacattttgcgacaccatgtggtcacgctgggtatttaagcccgagtgagcacgcagggtctccattttgaagcgggaggtttgaacgcgcagccgcc

SEQ ID NO: 7 - DNA, 163 bp, adeno-associated virus 2
gtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtc

SEQ ID NO: 8 - DNA, 45 bp, adeno-associated virus 2
ggaggggtggagtcgtgacgtgaattacgtcatagggttagggag

SEQ ID NO: 9 - DNA, 352 bp, adeno-associated virus 2
gtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccg

SEQ ID NO: 10 - DNA, 130 bp, adeno-associated virus 2; identical to SEQ ID NO: 1

SEQ ID NO: 11 - DNA, 397 bp, adeno-associated virus 2
ggaggggtggagtcgtgacgtgaattacgtcatagggttagggaggtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccg

SEQ ID NO: 12 - DNA, 208 bp, adeno-associated virus 2
ggaggggtggagtcgtgacgtgaattacgtcatagggttagggaggtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtc

SEQ ID NO: 13 - DNA, 1866 bp, adeno-associated virus 2
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaatag

SEQ ID NO: 14 - DNA, 2559 bp, adeno-associated virus 2
cagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 15 - protein, 735 aa, adeno-associated virus 2 [sequence omitted]

SEQ ID NO: 16 - DNA, 19 bp, Artificial Sequence
  TetR binding site
tccctatcagtgatagaga

SEQ ID NO: 17 - DNA, 107 bp, Artificial Sequence
  Modified MLP
cgccctcttcggcatcaaggaaggtgattggtttgtaggtgtaggccacgtgaccgggtgttcctgaaggggggctataaaaggtccctatcagtgatagagactca

SEQ ID NO: 18 - DNA, 107 bp, Artificial Sequence
  Modified MLP
cgccctcttcggcatcaaggaaggtgattggtttgtaggtgtaggccacgtgactccctatcagtgatagagaactataaaaggtccctatcagtgatagagactca

SEQ ID NO: 19 - protein, 137 aa, Human adenovirus D serotype 9 (HAdV-9)
MPRKKQEPLVEEMEEEWDSQAEEDEWEEETEEEELEEVEEEQATEQPVAAPSAPAAPAVTDTTSAAPAKPPRRWDRVKGDGKHERQGYRSWRAHKAAIIACLQDCGGNIAFARRYLLFHRGVNIPRNVLHYYRHLHS

SEQ ID NO: 20 - DNA, 585 bp, adenovirus 5
atggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatag

SEQ ID NO: 21 - protein, 194 aa, adenovirus 5
MAPKKKLQLPPPPTDEEEYWDSQAEEVLDEEEEDMMEDWESLDEEASEVEEVSDETPSPSVAFPSPAPQKSATGSSMATTSAPQAPPALPVRRPNRRWDTTGTRAGKSKQPPPLAQEQQQRQGYRSWRGHKNAIVACLQDCGGNISFARRFLLYHHGVAFPRNILHYYRHLYSPYCTGGSGSNSSGHTEAKATG

SEQ ID NO: 22 - DNA, 690 bp, adenovirus 5
atggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccgcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactag

SEQ ID NO: 23 - protein, 229 aa, adenovirus 5
MAPKKKLQLPPPPTDEEEYWDSQAEEVLDEEEEDMMEDWESLDEEASEVEEVSDETPSPSVAFPSPAPQKSATGSSMATTSAPQAPPALPVRRPNRRWDTTGTRAAHTAPAAAAAAATAAATQKQRRPDSKTLTKPKKSTAAAAAGGGALRLAPNEPVSTRELRNRIFPTLYAIFQQSRGQEQELKIKNRSLRSLTRSCLYHKSEDQLRRTLEDAEALFSKYCALTLKD

SEQ ID NO: 24 - DNA, 220 bp, adeno-associated virus 2
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccc

SEQ ID NO: 25 - DNA, 33 bp, adeno-associated virus 2
atgccggggttttacgagattgtgattaaggtc